SEQUENCE LISTING (7 sequences)

SEQ ID NO: 1 - DNA, 20 bp, synthetic construct
  source 1..20: mol_type = other DNA
tgcgtcgcgccatggcggcc

SEQ ID NO: 2 - DNA, 20 bp, synthetic construct
  source 1..20: mol_type = other DNA
gatggtctgatagccggcgt

SEQ ID NO: 3 - DNA, 25 bp, synthetic construct
  source 1..25: mol_type = other DNA
atgaagcccaaactgatgtaccagg

SEQ ID NO: 4 - DNA, 18 bp, synthetic construct
  source 1..18: mol_type = other DNA
tcaaagcaggcggcaggc

SEQ ID NO: 5 - RNA, 20 nt, synthetic construct
  source 1..20: mol_type = other RNA
gtcctgatcctggcggtagt

SEQ ID NO: 6 - RNA, 20 nt, synthetic construct
  source 1..20: mol_type = other RNA
gctagtggaggggttgctcg

SEQ ID NO: 7 - RNA, 18 nt, synthetic construct
  source 1..18: mol_type = other RNA
ggaagagcgagctcttct

(19) United States
(12) Patent Application Publication
(10) Pub. No.: US 2024/0424142 A1
(43) Pub. Date: Dec. 26, 2024

(54) METHODS OF TREATING NEURODEGENERATIVE CONDITIONS AND COMPOSITIONS THEREFOR

(71) Applicant: Yale University, New Haven, CT (US)

(72) Inventors: Jaime GRUTZENDLER, New Haven, CT (US); Peng YUAN, New Haven, CT (US); Mengyang ZHANG, New Haven, CT (US); Lei TONG, New Haven, CT (US)

(21) Appl. No.: 18/689,161

(22) PCT Filed: Sep. 7, 2022
(86) PCT No.: PCT/US2022/076012

§ 371 Date: Mar. 5, 2024

Related U.S. Application Data
(60) Provisional application No. 63/241,140, filed on Sep. 7, 2021.

(51) Int. Cl. A61K 48/00; A61P 25/28; C12N 9/22; C12N 15/11; C12N 15/90

(52) U.S. Cl. CPC A61K 48/0058; A61P 25/28; C12N 9/22; C12N 15/11; C12N 15/907; C12N 2310/20; C12N 2750/14143

(57) ABSTRACT

Described herein is a method of reversing or preventing a formation or enlargement of an axonal spheroid. The method includes introducing into a neuron affected by formation or enlargement of the axonal spheroid a compound that downregulates PLD3 expression level or activity. Also described herein is a method of treating or preventing a neurodegenerative condition in a subject in need thereof. The method includes administering to the subject a compound that downregulates PLD3 expression level or activity in a neuron cell affected by the neurodegenerative condition. Further described herein is a pharmaceutical composition for treating a neurodegenerative condition in a subject. The pharmaceutical composition includes a compound that downregulates PDL3 expression level or activity in a neuron cell affected by the neurodegenerative condition; and a pharmaceutically acceptable carrier.

### CROSS-REFERENCE TO RELATED APPLICATIONS

The present application claims priority under 35 U.S.C. § 119 (e) to U.S. Provisional Patent Application No. 63/241,140, filed Sep. 7, 2021, which is incorporated herein by reference in its entirety.

### STATEMENT REGARDING FEDERALLY SPONSORED RESEARCH

This invention was made with government support under NS089734 and AG058257 awarded by National Institutes of Health. The government has certain rights in the invention.

### SEQUENCE LISTING

The ASCII text file named “047162-7337WO1 (01756)_Seq Listing” created on Aug. 16, 2022, comprising 7.1 Kbytes, is hereby incorporated by reference in its entirety.

### BACKGROUND

Alzheimer's disease (AD) is a neurodegenerative condition characterized by widespread disruption in neural connectivity. The extracellular deposition of the beta-amyloid (Aβ) peptide is thought to trigger a cascade of events, eventually leading to cognitive decline. However, the cellular underpinnings linking Aβ deposition and neural network disruption are not well understood, limiting the rational design of new therapies. Extensive previous work has focused on mechanisms such as cell death and synapse loss as potential causes of neural dysfunction, and therapeutic efforts have mainly centered on strategies for extracellular amyloid removal.

There is thus a need in the art for novel composition and methods that can be used to treat, ameliorate, and/or prevent neurodegenerative conditions, such as but not limited to Alzheimer's disease. The present invention addresses this need.

### SUMMARY

In some embodiments, the present invention is directed to the following non-limiting embodiments:

Method of Reversing or Preventing Formation and/or Enlargement of Axonal Spheroid

In some embodiments, the present invention is directed to a method of reversing or preventing a formation and/or enlargement of an axonal spheroid.

In some embodiments, the method comprises: contacting a neuron affected by the formation or enlargement of the axonal spheroid with a compound that downregulates an expression level and/or an activity of PLD3.

In some embodiments, the axonal spheroid blocks or delays a propagation of an action potential (AP) along an axon of the neuron.

In some embodiments, the formation and/or enlargement of axonal spheroids is associated with a neurodegenerative condition in a subject.

In some embodiments, the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia.

In some embodiments, the neurodegenerative condition is Alzheimer's disease.

In some embodiments, the axonal spheroid is associated with an amyloid plaque.

In some embodiments, the compound that downregulates the expression level or the activity of PLD3 comprises a small molecule inhibitor of PLD3. In some embodiments, the compound that downregulates the expression level or the activity of PLD3 comprises a protein inhibitor of PLD3. In some embodiments, the compound that downregulates the expression level or the activity of PLD3 comprises a nucleic acid that downregulates the expression level and/or activity of PLD3 by RNA interference, and/or an expression vector expressing the nucleic acid that downregulates the expression level and/or activity of PLD3 by RNA interference. In some embodiments, the compound that downregulates the expression level or the activity of PLD3 comprises a ribozyme that downregulates the expression level and/or activity of PLD3, and/or an expression vector expressing the ribozyme. In some embodiments, the compound that downregulates the expression level or the activity of PLD3 comprises an expression vector comprising an expression cassette, wherein the expression cassette expresses CRISPR components that downregulate the expression level and/or activity of PLD3 by CRISPR knockout or CRISPR knockdown. In some embodiments, the compound that downregulates the expression level or the activity of PLD3 comprises a trans-dominant negative mutant protein of PLD3, and/or an expression vector that expresses the trans-dominant negative mutant protein of PLD3.

Method of treating, ameliorating, and/or preventing neurodegenerative condition

In some embodiments, the present invention is directed to a method of treating, ameliorating, and/or preventing a neurodegenerative condition in a subject in need thereof.

In some embodiments, the method comprises: administering to the subject a compound that downregulates an expression level and/or activity of PLD3.

In some embodiments, the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and/or Lewy Body dementia.

In some embodiments, the neurodegenerative condition is Alzheimer's disease.

In some embodiments, the subject is a human.

In some embodiments, the compound that downregulates the expression level and/or activity of PLD3 stops or reverses formation and/or enlargement of an axonal spheroid on an axon of a neuron cell.

In some embodiments the compound comprises a small molecule inhibitor of PLD3. In some embodiments the compound comprises a protein inhibitor of PLD3. In some embodiments the compound comprises a nucleic acid that downregulates the expression level and/or activity of PLD3 by RNA interference, and/or an expression vector expressing the nucleic acid that downregulates the expression level and/or activity of PLD3 by RNA interference. In some embodiments the compound comprises a ribozyme that downregulates the expression level and/or activity of PLD3, and/or an expression vector expressing the ribozyme. In some embodiments the compound comprises an expression vector comprising an expression cassette, wherein the expression cassette expresses CRISPR components that downregulate the expression level and/or activity of PLD3 by CRISPR knockout or CRISPR knockdown. In some embodiments the compound comprises a trans-dominant negative mutant protein of PLD3, and/or an expression vector that expresses the trans-dominant negative mutant protein of PLD3.

In some embodiments, the compound comprises at least one selected from the group consisting of: the expression vector expressing the ribozyme, the expression vector comprising an expression cassette expressing the CRISPR components, and the expression vector that expresses the trans-dominant negative mutant protein.

In some embodiments, the expression vector comprises a viral vector.

In some embodiments, the expression vector comprises an adeno-associated virus (AAV).

### Pharmaceutical Composition for Treating Neurodegenerative Condition

In some embodiments, the present invention is directed to a pharmaceutical composition for treating a neurodegenerative condition in a subject.

In some embodiments, the pharmaceutical composition comprises: a compound that downregulates an expression level and/or activity of PLD3 in a neuron cell affected by the neurodegenerative condition; and a pharmaceutically acceptable carrier.

In some embodiments, the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia.

In some embodiments, the neurodegenerative condition is Alzheimer's disease.

In some embodiments the compound comprises a small molecule inhibitor of PLD3. In some embodiments the compound comprises a protein inhibitor of PLD3. In some embodiments the compound comprises a nucleic acid that downregulates the expression level and/or activity of PLD3 by RNA interference, and/or an expression vector expressing the nucleic acid that downregulates the expression level and/or activity of PLD3 by RNA interference. In some embodiments the compound comprises a ribozyme that downregulates the expression level and/or activity of PLD3, and/or an expression vector expressing the ribozyme. In some embodiments the compound comprises an expression vector comprising an expression cassette, wherein the expression cassette expresses CRISPR components that downregulate the expression level and/or activity of PLD3 by CRISPR knockout or CRISPR knockdown. In some embodiments the compound comprises a trans-dominant negative mutant protein of PLD3, and/or an expression vector that expresses the trans-dominant negative mutant protein of PLD3.

In some embodiments, the compound comprises at least one selected from the group consisting of: the expression vector expressing the ribozyme, the expression vector comprising an expression cassette expressing the CRISPR components, and the expression vector that expresses the trans-dominant negative mutant protein.

In some embodiments, the expression vector comprises a viral vector.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-IR demonstrate that plaque-associated axonal spheroids block action potential propagation and disrupt interhemispheric connectivity. FIG. 1A depicts a representative confocal image of plaque-associated axonal spheroids (PAAS) in a 5×FAD mouse, in accordance with some embodiments. A single axon is labeled by GFP-expressing AAV2 virus. FIG. 1B shows an estimation of the total number of spheroid-affected axons around individual amyloid plaques. FIG. 1C depicts in vivo two-photon time lapse images of PAAS, labeled with AAV2-tdTomato. The location of the amyloid plaque is indicated with dashed lines. Despite presence of PAAS, the parent axon showed no evidence of degeneration. A subset of PAAS were dynamic (arrows) and others stable (asterisk) over a two-month interval. FIG. 1D depicts the schematics of electric stimulation and two-photon calcium imaging experiments for measuring axonal conduction. FIG. 1E depicts an example of GCaMP6f-labled axons with (FIG. 1E, left panel) and without (FIG. 1E, right panel) PAAS. Plots show example traces of calcium dynamics (10 Hz imaging frame rate) in regions of interest at both axonal sides of PAAS (top and bottom). Arrows with flash icon indicate the time of stimulation. Inserts show zoomed-in plots of the calcium transients (gray rectangles). Black dotted lines indicate exponential regressions of the rising phase. The vertical dotted lines show extrapolated spike time. FIG. 1F depicts example traces of complete conduction block at two sides of PAAS. Arrows with flash icon indicate the time of stimulation and asterisks mark the blocked calcium transients. FIG. 1G depicts the quantification of the differences in estimated spike times at the two axonal sides with respect to individual PAAS. N=10 axons without AS; N=21 axons with small AS; and N=8 axons with large AS, obtained from N=14 mice. Mann-Whitney tests were performed. The line chart of FIG. 1H depicts the estimated probability distribution of the degree of conduction disruption in PAAS-forming axons (details about simulations is described herein in reference to FIGS. 6A-6F). The Pie charts of FIG. 1H depicts the percentages for different types of conduction disruption patterns observed experimentally or by computational model prediction. FIG. 1I depicts the schematics of electric stimulation and two-photon calcium imaging experiments for measuring long-range axonal conduction. FIG. 1J depicts example traces of calcium dynamics (10 Hz imaging frame rate) in transcallosal axons imaged on the contralateral hemisphere. Arrows with flash icon indicate the time of stimulation. Inserts show zoomed-in plots of the calcium transients (gray rectangle). Black dotted lines indicate exponential regressions of the rising phase. The vertical dotted lines show extrapolated spike time. FIG. 1K depicts the quantification of the difference in stimulation time and estimated spike time in wildtype and 5×FAD mice, presented by individual axons (FIG. 1K, left chart) or mice (FIG. 1K, right chart). N=51 axons in WT mice (N=3); N=58 axons in 5×FAD mice (N=8). Mann-Whitney tests were performed for analysis by axons; and paired t-test were performed for analysis by mice. FIG. 1L: Schematic of the strategy for axonal electrical stimulation and two-photon voltage imaging of cell bodies to measure antidromic long-range axonal conduction. FIG. 1M: Example of voltage sensor ASAP3-labeled cell body. Blue line indicates the region of line scan (left panel) and example kymograph of two-photon line scan of ASAP3 sampled at 1 kHz, following a 10 Hz electrical stimulation (right panel). Bars under the right panel indicate stimulation and black arrows indicate action potential. FIG. 1N: Example traces generated from spatial integration of line scan images, comparing WT with 5×FAD mice. Bars indicate electrical stimulation. The electric current applied and the fast Fourier transform power (FFT) is indicated below each trace. FIG. 1O: Plot showing the probability of action potential generation (FFT power) for each cell at a defined current (individual dots). Insert shows 2 examples of the probability of action potential generation in single cells at various current stimulations, in WT and 5×FAD mice. FIG. 1P: Quantification of the currents needed for successful action potential conduction (50% probability) for each cell (individual dots) in WT and 5×FAD mice. N=25 cells from 2 WT mice; N=27 cells from 3 AD mice. Mann-Whitney tests were performed. FIG. 1Q: Quantification of the time interval between stimulation time and AP spike time in WT and 5×FAD mice. N=59 cells from 4 WT mice; N=62 cells from 4 AD mice. Mann-Whitney tests were performed. FIG. 1R: Comparison of rise times measured at the soma with either GcaMP6f or ASAP3, while stimulating contralateral axons (see FIG. 1L). Highlighted region between dashed line indicates the similarity between the approximate rise times for GcaMP6f and ASAP3.

FIGS. 2A-2P demonstrate that the accumulation of abnormally enlarged multivesicular bodies is associated with spheroid expansion and cognition decline, in accordance with some embodiments. FIG. 2A shows a confocal image of PAAS in a 5×FAD mouse brain showing a prominent halo of spheroids labeled by anti-LAMP-1 immunohistochemistry (“Lamp1”) around an amyloid plaque (“ThioflavinS”). The lower panel of FIG. 1A shows the zoomed-in picture from the PAAS labeled with the white dashed box. Arrows indicate enlarged LAMP1-positive multivesicular bodies (MVBs). FIG. 1B depicts the quantification of large MVB occurrence within PAAS at different ages in 5×FAD mice. N=3 mice for each age group. Each dot represents average measurements from 200 to 500 individual PAAS. Kruskal-Wallis test was performed. FIG. 2C depicts the quantification of PAAS areas with and without enlarged MVBs. N=3 mice from each group. Each pair of dots represents average measurements from 50 to 100 individual PAAS in the same mouse. Paired t-test was performed. The upper panel of FIG. 2D is an electron microscopy images of PAAS (the portion enclosed by solid line) in a 5×FAD mouse brain. The lower panels of FIG. 2D depicts two examples of zoomed-in images of MVBs (dashed boxes). FIG. 2E show confocal images of PAAS with high and low Cathepsin D contents. White dotted lines mark the perimeters of PAAS. FIG. 2F depicts the quantification of spheroid size as a function of Cathepsin D immunoreactivity levels. N=6 mice for each group; each pair of dots represents average measurement from 50 PAAS in the same mouse. Paired t-test was performed. FIG. 2G shows a confocal image of PAAS expressing the pH sensor SEpHluorin-mCherry in a 5×FAD mouse brain. FIG. 2H depicts the quantification of PAAS size as a function of pH. Neutral and acidic pH are defined by a threshold of red-green fluorescence ratio of 0.5. N=4 mice for each group; each pair of dots represents the average measurement from 50 PAAS in the same mouse. Paired t-test was performed. FIG. 2I show confocal images of PAAS labeled by immunolabeling of V0A1 (ATPase H+ Transporting V0 Subunit A1) in a post-mortem human AD brain. Panels on the right show zoomed-in example images of PAAS (one from the box on the left), with large MVBs indicated by arrows. FIG. 2J depicts the quantification of PAAS size as a function of presence of enlarged MVBs. N=4 subjects from each group. Each dot represents the average measurements of 200 to 500 individual PAAS. Paired t-test was performed. FIG. 2K shows confocal images of PAAS labeled by APP and Cathepsin D immunohistochemistry in a post-mortem human AD brain. Right panels show zoomed-in examples of PAAS with low or high Cathepsin D contents. White dotted lines indicate the outlines of PAAS. FIG. 2L depicts the quantification of PAAS size as a function of Cathepsin D contents. N=11 human subjects. Each pair of dots represents average measurements from 50 PAAS in the same postmortem brain. Paired t test was performed. FIGS. 2M-2O depict the comparisons of PAAS features between AD and MCI patients (m, PAAS area; n, MVB occurrence; and o, Cathepsin D contents). Each dot represents average measurement from 50 PAAS in FIGS. 2M and 2O, 200˜500 PAAS in FIG. 2N. N=12 AD and 6 MCI subjects in FIGS. 2M and 2O; N=3 AD and 3 MCI subjects in FIG. 2N. Mann-Whitney tests were performed. FIG. 2P depicts the receiver operating characteristic (ROC) curves clearly differentiate AD from MCI patients using PAAS diameter and APP or Cathepsin D contents as parameters.

FIGS. 3A-3N demonstrates that PLD3 mediates multivesicular body enlargement and spheroid expansion, in accordance with some embodiments. FIG. 3A depicts two PLD3 immunohistochemistry images, both of which show marked enrichment of PLD3 in PAAS in human postmortem AD and 5×FAD brain tissue. FIG. 3B are a confocal (FIG. 3B, left panel) and expansion microscopy (FIG. 3B, right panel) images of PLD3 immunohistochemistry and LAMP1-GFP in PAAS. Arrows indicate PLD3 puncta in enlarged LAMP1-positive multivesicular bodies (MVBs). FIG. 3C depicts confocal images of PAAS in 5×FAD mice with PLD3 or control GFP AAV2-mediated overexpression. Right panels show zoomed-in examples. FIG. 3D depicts the quantification of PAAS area in 10-month-old 5×FAD mice with PLD3 or control GFP AAV2-mediated overexpression. N=3 and 5 mice for GFP and PLD3 groups, respectively. Each dot represents average measurements from 350 to 600 individual PAAS. Mann-Whitney tests were performed. FIG. 3E is a confocal image of adjacent PAAS with (dashed line) and without (white solid line) PLD3 overexpression. Arrows indicate enlarged MVBs. FIG. 3F depicts the quantification of enlarged MVB occurrence in PAAS of 10-month-old 5×FAD mice with PLD3 or control GFP overexpression. N=3 and 4 mice for GFP and PLD3 groups, respectively. Each dot represents average measurement from 150 to 200 individual PAAS. Mann-Whitney tests were performed. FIG. 3G are confocal images of PAAS in 5×FAD mice with PLD3 or control GFP overexpression. Arrows indicate enlarged MVBs. FIG. 3H depicts the quantification of MVB size in PAAS of 10-month-old 5×FAD mice with PLD3 or control GFP overexpression. N=3 and 4 mice for GFP and PLD3 group, respectively. Each dot represents average measurement from 500 to 1000 MVBs. Mann-Whitney tests were performed. FIG. 3I shows confocal (left two images) and expansion microscopy (right two) images of Aβ42 immunohistochemistry (“Abeta42”) and LAMP1-GFP in PAAS. Arrows indicate Aβ42 puncta in enlarged LAMP1-positive MVBs. FIG. 3J show confocal images of FM1-43 dye (endocytosis marker) incorporation into PAAS in cultured brain slices following vehicle or PitStop2 (endocytosis inhibitor) treatment. FIG. 3K depicts the quantification of FM1-43 incorporation into PAAS with PitStop or dynasore treatment. N=20 PAAS for PitStop2 or dynasore at different concentrations. Data are represented as mean±S.E.M. Red dash lines show regression to a sigmoid inhibition curve. F-tests were used to compare the fitted top and bottom parameters for each group. FIG. 3L depicts the schematics of in vivo assay of intra parenchymal brain microinjections of fluorescently labeled Aβ-42 peptide for measuring Aβ endocytosis into PAAS. FIG. 3M show confocal images of injected fluorescently tagged Aβ-42 (“Injected Aβ”) incorporated into PAAS. The dashed lines indicate the outline of PAAS based on LAMP-1 immunohistochemistry. Arrows point to Aβ-42 puncta. FIG. 3N depicts the quantification of Aβ-42 incorporation in PAAS. N=3 mice, each with average measurements from 10 field of view, Wilcoxon matched-pairs signed rank tests were used to compare between groups.

FIGS. 4A-4J demonstrate that CRISPR/Cas9-mediated PLD3 deletion reduces PAAS size and improves axonal conduction, in accordance with some embodiments. FIG. 4A depicts schematics of two guide RNAs targeting the PLD3 gene. FIG. 4B show confocal images of adjacent PAAS with (GFP+) and without (GFP−) PLD3 deletion. Dashed lines mark the outlines of individual PAAS. Arrows indicate enlarged MVBs. FIG. 4C depicts the quantification of MVB occurrence in PLD3-deleted and control PAAS in 10-month-old 5×FAD/LSL-Cas9 mice. N=4 mice for each group. Each dot is the average of 150 to 250 individual PAAS measurements. Mann-Whitney tests were performed. FIG. 4D shows confocal images of PAAS expressing control scrambled sgRNAs (upper two images) or PLD3-targeting sgRNAs (lower two images) in 5×FAD/LSL-Cas9 mice, showing infected (GFP+) and uninfected (LAMP-1 immunohistochemistry) PAAS near a plaque (cyan). FIG. 4E depicts the quantification of PAAS sizes in 10-month-old mice with or without PLD3 deletion. N=6, 6, 4 mice for control sgRNA, PLD3 sgRNA-1 and PLD3 sgRNA-2, respectively. Each dot represents the average of 350 to 600 individual PAAS measurements. Mann-Whitney tests were performed. FIG. 4F depicts schematics of calcium imaging to measure conduction in contralateral axons with (yellow) or without (green) PLD3 manipulation. FIGS. 4G and 4I depicts example traces of calcium dynamics (20 Hz imaging frame rate) in contralateral axons following PLD3 deletion with sgRNA-2 (FIG. 4G) or PLD3 overexpression (FIG. 41). Arrows with flash icon indicate the time of stimulation. Inserts show zoomed-in plots of the calcium transients (gray rectangle). Black dotted lines indicate exponential regressions of the rising phase and vertical green/orange/purple dashed lines show extrapolated spike times. FIGS. 4H and 4H depict quantification of the spike times in PLD3-manipulated and control axons, shown by either individual axons or individual mice. N=80 manipulated and 61 control axons, from N=6 mice with PLD3 sgRNA-2; N=69 manipulated and 49 control axons, from N=5 mice with PLD3 overexpression. Mann-Whitney tests were performed for analysis by axons; paired t-test were performed for analysis by mice.

FIGS. 5A-5E demonstrate that Amyloid plaque-associated spheroids are axonal in origin and show predominantly structural stability and some dynamisms over extended intervals, in accordance withs some embodiments. FIG. 5A show a confocal image of a coronal section of a mouse brain 4 weeks after receiving a unilateral subarachnoid injection of AAV2-GFP shows that only cell bodies on one hemisphere are GFP positive. Dashed box indicates a region of interest on the contralateral hemisphere where plaques and axons were imaged (zoomed images in FIG. 5B). FIG. 5B show zoomed images of a plaque showing spheroid structures that can only come from transcallosal projecting axons (“GFP”) and are not associated with the dendritic marker MAP2 immunolabeling (“MAP2”). FIG. 5C depicts quantification of the changes in axon spheroid number at different time intervals from in vivo time lapse images of individual axons, labeled with AAV-tdTomato (see FIGS. 1A-1K). Each dot indicates an axon. Arrow pointed dots indicate observed spheroid disappearance events. FIG. 5D show pie charts representation of data in FIG. 5C, showing the proportions of imaged axons that showed PAAS appearance, disappearance, or no change during the respective time intervals. FIG. 5E depicts the quantification of PAAS size change over time in individual axonal segments traced by in vivo imaging. Each line indicates a single axon.

FIGS. 6A-6F explain the computational modeling of axonal conduction abnormalities caused by PAAS, in accordance with some embodiments. FIG. 6A depicts computer simulations of membrane potentials recorded at two points on each side of PAAS (green and magenta arrows in upper panels) during a single action potential. Three different scenarios are presented demonstrating PAAS size-dependent conduction delays (lower panels) (details of the modeling results are described elsewhere herein). FIG. 6B depicts computer simulation of membrane potentials recorded at two points on each side of PAAS (the two arrows) during a 20 Hz stimulation train. While single action potentials can be completely blocked by larger PAAS, repetitive stimulation can eventually lead to successful conduction of the action potential due to a capacitor effect of PAAS (discussed elsewhere herein). FIGS. 6C-6D depict modeling of a simple resistor-capacitor electric circuit with 3 different levels of capacitance. Dashed line indicates 3 volts as an arbitrary threshold mimicking the minimal membrane potential to trigger neuronal firing. FIG. 6E depicts the representation of the simulation results with a range of spheroid diameters and membrane ion channel densities. FIG. 6F depicts the frequency distribution of the number of spheroids per individual axon (top graph) and logarithmic transformation of the diameters of individual spheroids (bottom graph) quantified from confocal images of virally labeled individual axons in 5×FAD mice, showing a gaussian distribution (D'Agostino & Pearson normality test >0.05, n=76 for bulb number and 382 for bulb size. The fitted gaussian curves, 25 and 75 percentile values are marked with red lines).

FIGS. 7A-7D demonstrate that axonal spheroids markedly disrupt spontaneous action potential conduction, in accordance with some embodiments. FIG. 7A depicts two-photon in vivo calcium imaging of spontaneous activity in axons near amyloid plaques (white arrow) with and without PAAS. Given the lower frequency of spontaneously active neurons, lower frame rates were used to image larger fields of view and were thus unable to measure precisely the Ca²⁺ rise times like in FIGS. 1A-1K. FIG. 1B depicts example traces of GCaMP6s fluorescence signal obtained from ROIs (the circles in FIG. 7A) at the two sides of the plaques indicated in FIG. 7A (2 Hz imaging frame rate). Mismatched Ca²⁺ transients are indicated with arrows. FIG. 7C shows correlation maps calculated using the average fluorescence intensity within ROII (left circles in FIG. 7A, as reference, and color-coded for correlation coefficient to every other pixel within the field of view. FIG. 7D depicts the quantification of the decorrelation of GCaMP6s fluorescence in ROIs at the two axonal sides with respect to the plaque, during spontaneous Ca²⁺ transients (n=12, axons without PAAS; and n=10, axons with PAAS). Mann-Whitney test was used for comparison.

FIGS. 8A-8F demonstrate that PAAS number correlates with severity of cognitive decline in humans, in accordance with some embodiments. FIG. 8A shows axon spheroids labeled by Amyloid Precursor Protein (APP) immunohistochemistry in post-mortem human brain (middle frontal gyrus), from subjects with mild cognitive impairment (MCI) and AD. FIG. 8B depicts the quantification of total spheroid number around individual plaques based on confocal images of APP immunofluorescence. Each dot indicates the average from 25 plaque measurements of an individual subject. Bars indicate group average. Mann-Whitney tests were used for comparisons. N=6 MCI and 12 AD subjects. FIG. 8C: Quantification of total spheroid number around individual plaques. Each dot indicates the average of 25 plaque measurements of an individual subject. Bars indicate group average. Mann-Whitney tests were used for comparisons. N=12 AD and 6 MCI subjects. Quantification of PAAS size in AD and MCI patients based on confocal images of APP immunofluorescence. Each dot represents an average measurement of 50 PAAS. N=12 AD and 6 MCI subjects. Mann-Whitney tests were performed. FIG. 8D: Quantification of PAAS size in AD and MCI patients based on confocal images of VOA 1 immunostaining. Each dot represents an average measurement of 200˜250 PAAS. N=6 AD and 4 MCI patients. Mann-Whitney tests were performed. FIG. 8E lists the ApoE genotype, age, and Braak stage information of all the human brain tissue used in this study. FIG. 8F: CDR score and ABC score of human AD with PLD3 V232M variant brain tissue used in this study.

FIGS. 9A-9D demonstrate that no PLD3 protein expression in microglia or astrocytes in 5×FAD mice or human AD brain, in accordance with some embodiments. FIGS. 9A and 9B are confocal images showing absence of PLD3 signal (“PLD3”) within Iba1-labeled microglia (“Iba1”) in 5×FAD mouse brain (FIG. 9A) and postmortem brain tissue of AD human patients (FIG. 9D). FIG. 9C shows confocal images of 5×FAD mouse brain, which show absence of PLD3 signal (“PLD3”) within S100-labeled astrocyte (“S100”). FIG. 9D show confocal images of human AD postmortem brain tissue, which show absence of PLD3 signal (“PLD3”) within ALDHIL1-labeled astrocyte (“ALDHIL1”).

FIGS. 10A-10I describe additional analyses of Multivesicular bodies, spheroids and amyloid plaques in 5×FAD mice with PLD3 over expression, in accordance with some embodiments. FIG. 10A show confocal images of virus infected (“GFP”) and uninfected (“Lamp1”) PAAS in 5×FAD mice with control GFP or PLD3 overexpression. FIG. 10B depicts the quantification of PAAS sizes in 5-month-old 5×FAD mice with PLD3 or GFP overexpression. N=6 and 5 mice for PLD3 and GFP groups, respectively. Each dot represents average from 350˜600 PAAS measurements. Mann-Whitney tests were performed. FIG. 10C depicts the quantification of large MVBs occurrence in PAAS in 5-month-old 5×FAD mice with PLD3 or GFP overexpression. N=4 mice for each group. Each dot represents average measurement from 150˜250 PAAS. Mann-Whitney tests were performed. FIG. 10D shows zoomed-in example images of PAAS with and without PLD3 overexpression. The dash lines mark the outline of individual PAAS. Arrows indicated enlarged MVBs. FIGS. 10E-10F depict the quantification of plaque number (FIG. 10E) and size (FIG. 10F) in mice with GFP or PLD3 overexpression. N=5 and 6 for GFP and PLD3 groups, respectively. For FIG. 10F, each dot represents average from 100˜250 plaque measurements. Unpaired t-tests were performed. FIG. 10G shows confocal images of LAMP1-positive vesicular structures in PAAS and cell bodies in 10-month-old mice with PLD3 overexpression. FIG. 10H depicts the quantification of MVB sizes in groups described in FIG. 10G. N=4 mice for each group. Each dot represents average measurement from 500˜1000 MVBs from PAAS or 100-200 LAMP1-positive vesicles from cell bodies. Welch's t-test was used. FIG. 10I shows confocal images of spheroids and LAMP1-positive vesicles in wildtype mice overexpressing PLD3.

FIGS. 11A-11F describe the validation of CRISPR-Cas9-mediated PLD3 deletion, in accordance with some embodiments. FIGS. 11A-11D are confocal images of PLD3 immunohistochemistry in tissue infected (“GFP”) and uninfected with PLD3-targeted sgRNA1 (FIGS. 11A and 11C) or sgRNA2 (FIGS. 11B and 11D). Dashed lines indicate outlines of infected cell bodies or individual spheroids. Rectangles defined by dashed lines shown in FIGS. 11A and 11B indicate zoomed-in field of views on the right. FIGS. 11E-11F depict the quantifications of PLD3 fluorescence intensities in cell bodies with (GFP+) or without (GFP−) PLD3-targeted sgRNA1 (FIG. 11E) or sgRNA2 (FIG. 11F). 15˜25 cell bodies were measured from each group. Mann-Whitney tests were performed.

FIGS. 12A-12F describe additional analyses of multivesicular bodies, spheroids and amyloid plaques in mice with PLD3 deletion, in accordance with some embodiments. FIGS. 12A-12B depict confocal images of infected and uninfected PAAS in 5×FAD mice with scrambled sgRNA (FIG. 12A) or PLD3 targeted sgRNA (FIG. 12B). FIG. 12C depicts the quantification of PAAS area in 5-month-old mice with control sgRNA or PLD3 sgRNA 2. N=4 and 5 mice for control and PLD3 sgRNA groups, respectively. Each dot represents average from 350˜600 PAAS measurements. Mann-Whitney tests were performed. FIG. 12D depicts the quantification of MVBs occurrence in mice with control sgRNA or PLD3 sgRNA 2. N=4 mice for each group. Each dot represents average from 150˜250 PAAS measurements. Mann-Whitney tests were performed. FIG. 12E-12F depict the quantification of plaque number (FIG. 12E) and size (FIG. 12F) in mice with control or PLD3 sgRNA. N=5 mice for each group. Each dot represents average from 100˜250 plaque measurements. Unpaired t-tests were performed.

FIGS. 13A-13D describe additional analyses of axonal conduction upon PLD3 modulation, in accordance with some embodiments. FIG. 13A depicts the quantification of the difference in stimulation time and estimated spike time in axons with scrambled sgRNA and control GCaMP only axons in 5×FAD mice, presented by individual axons or by mice. n=69 manipulated and 37 control axons, and N=4 mice. FIG. 13B depicts the quantification of the difference in stimulation time and estimated spike time in axons with dTomato overexpression and control GCaMP only axons in 5×FAD mice, presented by individual axons or by mice. n=42 manipulated and 21 control axons, and N=4 mice. FIG. 13C shows example traces of calcium dynamics (20 Hz imaging frame rate) in axons of imaging hemispheres in PLD3 deletion with sgRNA 1. Arrows with flash icon indicate the time of stimulation. Inserts show zoomed-in plots of the calcium transients (gray blocks). Black lines indicate exponential regressions of the rising phase and dotted lines show extrapolated spike time. FIG. 13D depicts the quantification of the difference in stimulation time and estimated spike time in manipulated and control axons in 5×FAD mice, presented by individual axons or by mice. n=71 manipulated and 46 control axons, and N=5 mice. Mann-Whitney tests were performed.

FIG. 14 depicts the proposed model of PAAS enlargement and functional consequences in Alzheimer's disease, in accordance with some embodiments. 1) The present study demonstrated that the accumulation of abnormally enlarged MVBs is a major driver of PAAS enlargement. Small PAAS predominately contain mature lysosomes, while bigger PAAS contain abundant and enlarged MVBs. 2) The present study identified PLD3 as a critical modulator of MVB abnormalities and subsequent spheroid enlargement. PLD3 is uniquely sorted through the ESCRT pathway into the intralumenal vesicles (ILVs) of MVBs. Accumulation of PLD3 at spheroids could lead to MVB enlargement by interfering with ESCRT machinery. This process could be exacerbated with the presence of Aβ. Aβ from extracellular amyloid deposits is actively endocytosed and is present in the same subcellular compartments as PLD3. PLD3 could thus work synergistically with Aβ, leading to greater MVB abnormalities. 3) Large PAAS cause more severe conduction blocks, by functioning as electrical capacitors that act as current sinks. Given that hundreds of axons around each plaque develop spheroids and these structures remain stable for extended periods of times, the large number of plaques present in the AD brain could significantly affect neural networks by widespread disruption of axonal connectivity. 4) The present study found that cortical neurons in 5×FAD mice exhibited hyperactivity, and this can be corrected by restoring axon conduction through reducing PAAS in basal forebrain cholinergic projections. This suggests that PAAS can cause widespread disruption of neural circuit function. In addition, parallel compact axonal bundles that follow a stereotyped projection path along a tri-synaptic loop in hippocampus, a region critical for memory formation, could be particularly vulnerable to amyloid plaques located in the region. Furthermore, neural processes that rely on temporally precise long-range coordination among brain regions, such as memory consolidation, could be severely affected. In addition, synaptic plasticity could also be disrupted, due to the requirement of precise timing of firing between pre-synaptic and post-synaptic terminals. Altogether, action potential blocks caused by PAAS could be detrimental to various neural processes such as memory formation and reaction time, potentially contributing to cognitive decline in AD.

FIG. 15 depicts the estimated size distribution of PAAS according to some embodiments.

FIG. 16 depicts the relationship between the total volume of PAAS with the number of axons according to some embodiments.

FIG. 17 depicts the relationship between the number of axons per plaque and the plaque diameters according to some embodiments.

FIG. 18 depicts an electric circuit including a simple capacitor that charges or discharges according to some embodiments.

FIG. 19 depicts the effects of the capacitor on the electric circuit as depicted in FIG. 18 when the pulse generator gives a single pulse according to some embodiments. The voltages as depicted in FIG. 19 are voltages on the two ends of the capacitor of FIG. 18 changing over time.

FIG. 20 depicts the effects of the capacitor on the electric circuit as depicted in FIG. 18 when the pulse generator gives a 10 Hz signal according to some embodiments.

FIGS. 21A-21G demonstrates that the reduction in axonal spheroids by PLD3 deletion improves neural circuit function, in accordance with some embodiments. FIG. 21A: Schematics showing cholinergic neurons in the basal forebrain projecting to the cortex following infection with AAV viruses encoding either PLD3 or control sgRNAs (left panel), and two photon images show intermingled projecting axons from basal forebrain (“tdTomato”) with GCaMP6f-labeled cortical neurons (right panel). Calcium imaging was performed in cortical neurons of awake mice in the same region as the projecting forebrain axons which are likely cholinergic. FIG. 21B: Representative two-photon image of GCaMP6f-labeled cortical neurons. FIG. 21C: Example of raw calcium traces from selected individual cortical neurons. FIG. 21D: Quantification of spike counts from individual neurons during a 30-minute imaging session. Each dot represents the average spike count from all cells in the same mouse. Bars indicate group mean. Violin plots show distributions of spike counts from all individual neurons from the same group. FIG. 21E: Quantification of pair-wise mutual information grouped by distances between neurons. Two-way ANOVA test was used to compare between groups. FIG. 21F: Quantification of neurons classified in clusters by their activity patterns (Louvian clustering, see methods) and represented by cluster size distribution. FIG. 21G: Quantification of population entropy (as a measurement of temporal variance of the firing pattern) from each mouse imaged. For FIGS. 21D, 21E and 21G, N=4, 4, and 6 mice in wildtype group, 5×FAD with control sgRNA group and 5×FAD with PLD3 sgRNA group, respectively. For FIG. 21F, N=67, 21, and 45 clusters in wildtype group, 5×FAD with control sgRNA group and 5×FAD with PLD3 sgRNA group, respectively. For FIGS. 21D, 21F and 21G, One-way ANOVA test was used to compare among groups and p-values indicating the post-hoc comparison between groups, with Sidak's correction for multiple comparison.

FIG. 22 is a diagram explaining the difference in measurements in interhemispheric conduction with antidromic versus orthodromic stimulations, in accordance with some embodiments.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

In one aspect, the present invention provides compositions and methods for treating, ameliorating, and/or preventing certain neurodegenerative diseases and/or disorders, such as but not limited to Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and/or Lewy Body dementia. In certain embodiments, the method comprises administering to the subject in need thereof a therapeutically effective amount of a compound that reverses and/or prevents formation and/or enlargement of an axonal spheroid. In certain embodiments, the axonal spheroid blocks or delays a propagation of an action potential (AP) along an axon of the neuron. In another aspect, the present invention provides compounds and methods for reversing and/or preventing formation and/or enlargement of an axonal spheroid in a cell, such as but not limited to a cell within a subject, such as but not limited to a subject suffering from and/or at risk of suffering a neurodegenerative disease.

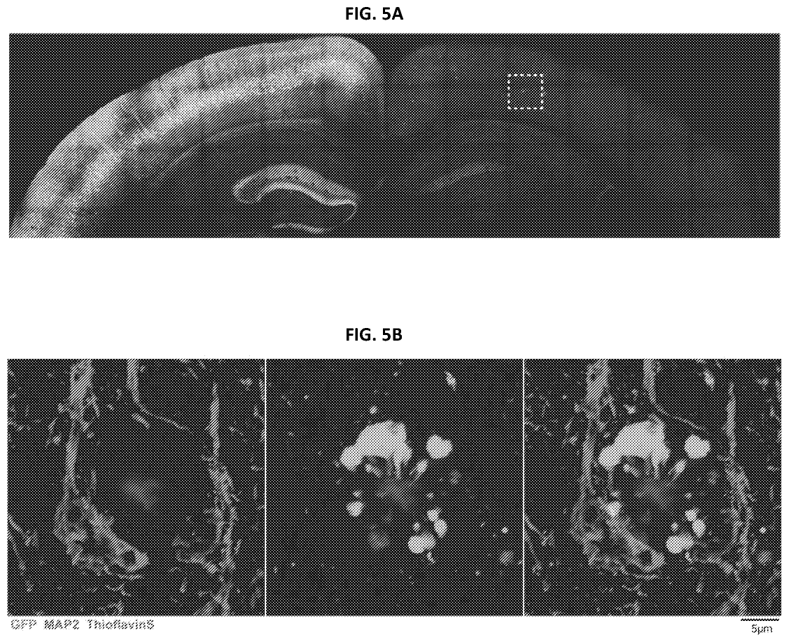

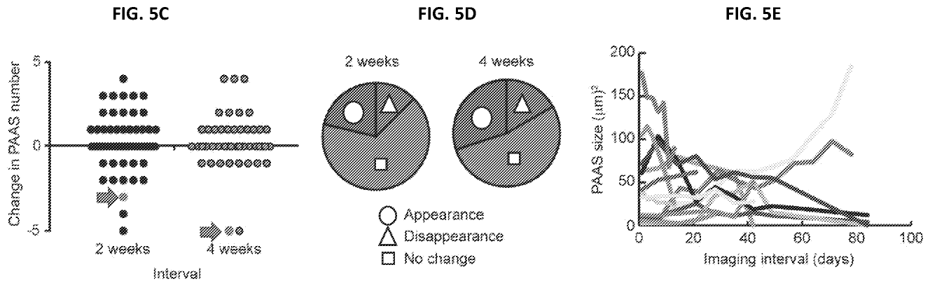

A significant and understudied pathological hallmark of Alzheimer's disease (AD) are the markedly enlarged neuronal processes, sometimes termed dystrophic neurites, found around Aβ deposits. These processes appear to be all axonal rather than dendritic in origin (FIGS. 5A-5B), and are therefore sometimes referred to as “plaque-associated axonal spheroids (PAAS)” herein. Although various hypotheses regarding their development have been proposed through the years, these structures have not been a major focus of therapeutics and their pathophysiological significance remains uncertain.

In one aspect, the present study identified the amyloid plaque-associated axonal spheroids as prominent contributors to neural network dysfunction, at least because the enlarging spheroids can act as action potential conduction blockades by causing electric current to sink in a size-dependent manner.

The present study demonstrates, using AD-like (Alzheimer's disease-like) mice as a model, that spheroid growth was driven by an age-dependent accumulation of large multivesicular bodies (MVBs) and was mechanistically linked with Phospholipase D3 (PLD3), a lysosomal protein and potential AD risk factor, which is sorted to MVBs and is highly enriched in axonal spheroids.

The present study further demonstrates that neuronal overexpression of PLD3 led to accumulation of MVBs and spheroid enlargement, which was associated with worsening of conduction blockades, while PLD3 deletion reduced MVBs and spheroid size, leading to marked improvement in long-range axonal conduction. Thus, modulation of neuronal MVB biogenesis through PLD3 could potentially reverse neural network abnormalities in AD, independently of amyloid removal.

As described elsewhere in this specification, a multipronged approach was undertaken to investigate PAAS, using high-resolution time-lapse intravital structural and calcium (Ca²⁺) imaging, expansion microscopy, single axon molecular manipulations and computational modeling. It was found that hundreds of axons develop PAAS around each amyloid deposit and rather than being structures associated with degenerative retracting axons, they are persistent and undergo dynamic changes in size over extended imaging intervals. PAAS markedly disrupt the propagation of action potentials (AP) by acting as electric current sinks, leading to abnormal interhemispheric connectivity. The degree of disruption in axonal conduction was found to be determined by the size of individual spheroids. The present study uncovered neuronal multivesicular body (MVB) biogenesis as a determinant of PAAS size, and identified the neuronal lysosomal protein PLD3 as a key modulator of MVB abnormalities and PAAS enlargement. These findings bring forward a new paradigm demonstrating that modulation of neuronal endo-lysosomal signaling and MVB biogenesis can have a major impact on axonal electrical conduction. Thus, targeting PAAS growth is a novel strategy for ameliorating network abnormalities in AD, without amyloid plaque removal.

In accordance with the above, the instant specification is, among others, directed to the following embodiments.

In some embodiments, the instant specification is directed to a method of reversing and/or preventing formation and/or enlargement of axonal spheroids. In some embodiments, the method includes contacting a neuron affected by the formation and/or enlargement of axonal spheroids with a compound that downregulates an expression level and/or activity of PLD3.

In some embodiments, the instant specification is directed to a method of treating, ameliorating, and/or preventing a neurodegenerative condition. In some embodiments, the method includes administering to a subject in need thereof a subject a compound that downregulates an expression level and/or activity of PLD3 in a neuron cell affected by the neurodegenerative condition. In some embodiments, the neurodegenerative condition is Alzheimer's disease.

In some embodiments, the instant specification is directed to a compound for treating, ameliorating, and/or preventing a neurodegenerative condition. In some embodiments, the compound downregulates an expression level and/or activity of PLD3 in a neuron cell affected by the neurodegenerative condition. In some embodiments, the neurodegenerative condition is Alzheimer's disease.

It is worth noting that, although the instant specification describes Alzheimer's disease as a non-limiting illustrative example, other types of neurodegenerative conditions are specifically included in the scope of the instant specification. One of ordinary skill in the art would understand that the prominent axonal pathology which includes the formation of large axonal enlargements (i.e., the spheroids) exists in other types of neurodegenerative conditions, such as but not limited to Lou Gehrig's disease (ALS), Huntington's disease, post traumatic encephalopathy, lysosomal storage disorders including Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and/or Lewy Body dementia, as well. As such, one of ordinary skill in the art would expect that the methods and compounds as described herein are applicable to other types of neurodegenerative conditions.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of +20% or +10%, in certain embodiments ±5%, in certain embodiments ±1%, in certain embodiments ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “treatment” or “treating” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a disease or disorder and/or a symptom of a disease or disorder, with the purpose to cure, heal, alleviate, relieve, alter, remedy, ameliorate, improve or affect the disease or disorder and/or the symptoms of the disease or disorder. Such treatments may be specifically tailored or modified, based on knowledge obtained from the field of pharmacogenomics.

### Method of Reversing or Preventing Formation or Enlargement of Axonal Spheroids

The formation of large axonal enlargements (i.e., the spheroids) is a neurological condition commonly found in many types of neurodegenerative conditions other than Alzheimer's disease. For example, Lou Gehrig's disease (ALS), Huntington's disease, post traumatic encephalopathy, lysosomal storage disorders including Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia are all known to involve axonal spheroids.

As detailed elsewhere in the instant specification, it was discovered that the formation and enlargement of axonal spheroids disrupt the propagation of action potentials (APs) along the axons. Using Alzheimer's disease as a model, it was discovered that axonal spheroids associated with amyloid plaques (also referred to as “plaque-associated axonal spheroids” or “PAAS” herein) disrupt the propagation of action potentials by acting as electric current sinks. The present study further provides methods of reducing the sizes of the axonal spheroids, and that doing so restores the axonal conduction properties of the axons affected by the axonal spheroids.

As detailed elsewhere in the instant specification, the present study demonstrated that downregulating PLD3 expression level reverses the enlargement of PAAS and restores the axonal conduction properties of the axons affected by the axonal spheroids, while overexpression of PLD3 has the opposite effects.

Therefore, in some embodiments, the instant specification is directed to a method of reversing or preventing a formation or enlargement of an axonal spheroid. In some embodiments, the axonal spheroid blocks or delays a propagation of an action potential (AP) along an axon of a neuron. In some embodiments, the method includes contacting a neuron affected by the formation and/or enlargement of axonal spheroids with a compound that downregulates an expression level and/or activity of PLD3. In some embodiments, the formation or enlargement of axonal spheroids is associated with a neurodegenerative condition. In some embodiments, the neurodegenerative condition is Alzheimer's disease, Lou Gehrig's disease (ALS), Huntington's disease, post traumatic encephalopathy, lysosomal storage disorders including Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, Lewy Body dementia, or combinations thereof. In some embodiments, the neurodegenerative condition comprises Alzheimer's disease. In some embodiments, the axonal spheroids are axonal spheroids associated with amyloid plaques.

In some embodiments, the compound that downregulates the expression level or the activity of PLD3 acts at the genomic level. For example, the expression level of PLD3 can be down-regulated by gene knockout, such as CRISPR knockout and other knockout techniques.

In some embodiments, the compound that downregulates the expression level or the activity of PLD3 acts at the transcriptional level or the translational level. For example, the expression level of PLD3 can be down-regulated by gene knockdown, such as by RNA interference technique, ribozyme knockdown, or CRISPR knockdown.

In some embodiments, the compound that downregulates the expression level or the activity of PLD3 acts at the post-translational level. For example, the expression level of PLD3 can be down-regulated by targeted protein degradation, such as proteolysis-targeting chimera (PROTAC) and other protein degradation strategies. For example, the activity of PLD3 can be down-regulated by small molecules inhibitors of PLD3, antibodies that neutralizes PLD3, and trans-dominant negative mutant of PLD3.

In some embodiments, the compound that downregulates the expression level or the activity of PLD3 includes a small molecule inhibitor of PLD3, a protein inhibitor of PLD3, or a compound that downregulates the expression level and/or activity of PLD3 by RNA interference, by ribozyme, by CRISPR knockout/knockdown, or by producing a trans-dominant negative mutant, and so forth.

In some embodiments, the compound contemplated herein can be delivered by a vector, such as a plasmid or a viral vector. One of ordinary skill in the art would understand that such vectors can be used to deliver compounds in the form of nucleic acids, such as RNA or DNA. Such vectors are described herein below.

In certain embodiments, the compound contemplated herein (including but not limited to nucleic acids) can be more efficiently delivered to the cell nucleus by coupling the compound with the monoclonal anti-DNA antibody 3E10, which penetrates living cells and localizes in the nucleus without causing any apparent harm to the cell (Hansen J E, et al., Intranuclear protein transduction through a nucleoside salvage pathway. J Biol Chem 2007; 282:20790-3; see also WO 2020/047353 and WO 2021/042060, all of which are incorporated herein in their entireties by reference). 3E10 and its single-chain variable fragment (3E10 scFv) have been developed as an intracellular delivery system for macromolecules. After localizing in the cell nucleus, 3E10 scFv is largely degraded within 4 hours, thus further minimizing any potential toxicity.

In certain embodiments, the compounds contemplated herein (including but not limited to nucleic acids) can be more efficiently delivered to the central nervous system using certain lipid nanoparticle formulations known in the art, such as but not limited to those described in Cullis, P. R. et al., Molecular Therapy Vol. 25 No 7 Jul. 2017. See also US20150165039 and WO 2014/008334, all of which are incorporated herein in their entireties by reference.

In certain embodiments, the compounds contemplated herein can be more efficiently delivered to tissue by coupling with certain protein fragments, called “pHLIP” (pH (Low) Insertion Peptide), which allow for the cargo to accumulate in acidic environments within the body. In certain embodiments, a polypeptide with a predominantly hydrophobic sequence long enough to span a membrane lipid bilayer as a transmembrane helix (TM) and comprising one or more dissociable groups inserts across a membrane spontaneously in a pH-dependent fashion placing one terminus inside cell. The polypeptide conjugated with various functional moieties delivers and accumulates them at cell membrane with low extracellular pH. The functional moiety conjugated with polypeptide terminus placed inside cell are translocated through the cell membrane in cytosol. The peptide and its variants or non-peptide analogs can be used to deliver therapeutic, prophylactic, diagnostic, imaging, gene regulation, cell regulation, or immunologic agents to or inside of cells in vitro or in vivo in tissue at low extracellular pH. See also US20080233107, WO2012/021790, US20120039990, US20120142042, US20150051153, US20150086617, and US20150191508, all of which are incorporated herein in their entireties by reference.

### Downregulating PLD3 by Small Molecule Inhibitors

In some embodiments, the compound that downregulates the expression level or the activity of PLD3 includes a small molecule that inhibits the activity of PLD3. As used herein, the term “small molecule” refers to a molecule having a size of less than 2000, 1800, 1600, 1400, 1200, 1000, 800, or 600 daltons.

Since PLD3 is a member of the member of the phospholipase D family, one of ordinary skill in the art would expect that many known phospholipase inhibitors could inhibit the activity of PLD3. Examples of small molecule phospholipase inhibitors includes clofazimine (also known as Lamprene or MNKD 101), RABI-767, MRX-4, MRX-6, and VEN 308. Further examples of small molecule phospholipase inhibitors includes PLD3 inhibitors cited in Shirey, et al., Bioorg. Med. Chem. Lett. 49 (2021): 128293 (incorporated herein in its entirety by reference).

In some embodiments, the small molecule inhibitor comprises a PROTAC or a Proteolysis Targeting Chimeric Molecule. PROTACs are heterobifunctional nanomolecules that can target any protein for ubiquitination and degradation. In certain embodiments, the PROTAC contemplated in the present invention comprises a group that is recognized by the E3 ubiquitin ligase and a group that is recognized by PLD3. The PROTAC is able to simultaneously bind to the PLD3 and the E3 ligase. Formation of such trimeric complex formation leads to the transfer of ubiquitins to the PLD3, marking it for degradation. PROTAC molecules possess good tissue distribution and the ability to target intracellular proteins, thus can be directly applied to cells or injected into animals without the use of vectors. PROTACS useful within the invention can be prepared using any known compound that binds to and/or recognizes and/or inhibits PLD3, which is linked through a linker to an E3 ubiquitin ligase, such as but not limited to those described in WO 2013/106643, WO 2013/106646, and WO 2019/148055.

### Downregulating PLD3 by Protein Inhibitors of PLD3

In some embodiments, the compound that downregulates the expression level or the activity of PLD3 includes a protein that downregulates the expression level or the activity of PLD3.

Since PLD3 is a member of the phospholipase family, one of ordinary skill in the art would expect that many proteins that are known to downregulate the expression level and/or activity of phospholipase could reduce the level activity of PLD3.

Examples of monoclonal and/or polyclonal antibodies that target PLD3 include SBI-3150, NBP1-59921 (Novus Biologicals, Centennial, CO), HPA012800 (Millipore Sigma, St Louis, MO), PA5-52985, PA5-42640, 17327-1-AP, PA5-104016, PA5-31959 (ThermoFisher, Waltham, MA), LS-C155704, LS-C216828 (LSBio, Seattle, WA), and any humanized derivatives thereof.

Examples of non-antibody proteins that inhibit phospholipases include CB-24 (Crotoxin), uteroglobins such as CG100, CG-201, CG367 and CG459, and VRCTC310 (Crotoxin and Cardiotoxin).

In some embodiments, the protein that downregulates the expression level and/or activity of PLD3 is administered in form of a protein. In some embodiments, the protein that downregulates the expression level and/or activity of PLD3 is administered in form of a nucleic acid that expresses the protein, such as an expression vector. The expression vector is described in the “Vector” section elsewhere in the instant specification.

### Downregulating PLD3 by RNA Interference

In some embodiments, the compound that downregulates the activity or expression level of PLD3 includes a nucleic acid that downregulates the activity and/or expression level of PLD3 by the means of RNA interreference.

In some embodiments, the nucleic acid that downregulates the expression level of PLD3 by the means of RNA interreference includes an isolated nucleic acid. In other embodiments, the modulator is an RNAi molecule (such as but not limited to siRNA and/or shRNA and/or miRNAs) or antisense molecule, which inhibits PLD3 expression and/or activity. In yet other embodiments, the nucleic acid comprises a promoter/regulatory sequence, such that the nucleic acid is preferably capable of directing expression of the nucleic acid. Thus, the instant specification provides expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, siRNA is used to decrease the level of PLD3. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391 (19): 306-311; Timmons et al., 1998, Nature 395:854; Montgomery et al., 1998, TIG 14 (7): 255-258; Engelke, Ed., RNA Interference (RNAi) Nuts & Bolts of RNAi Technology, DNA Press, Eagleville, PA (2003); and Hannon, Ed., RNAi A Guide to Gene Silencing, Cold Spring Harbor Laboratory Press, Cold Spring Harbor, NY (2003). Soutschek et al. (2004, Nature 432:173-178) describes a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the instant specification also includes methods of decreasing levels of PLD3 using RNAi technology.

In certain embodiments, the instant specification provides a vector comprising an siRNA or antisense polynucleotide. In other embodiments, the siRNA or antisense polynucleotide inhibits the expression of PLD3. The incorporation of a desired polynucleotide into a vector and the choice of vectors is well-known in the art.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In certain embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Following the generation of the siRNA polynucleotide, a skilled artisan will understand that the siRNA polynucleotide has certain characteristics that can be modified to improve the siRNA as a therapeutic compound. Therefore, in some embodiments, the siRNA polynucleotide is further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine.

In certain embodiments, an antisense nucleic acid sequence expressed by a plasmid vector is used to inhibit PLD3 protein expression. The antisense expressing vector is used to transfect a mammalian cell or the mammal itself, thereby causing reduced endogenous expression of PLD3.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the instant specification may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the instant specification include oligonucleotide derivatives known in the art which have improved biological activity compared to unmodified oligonucleotides (see U.S. Pat. No. 5,023,243).

### Downregulating PLD3 by Ribozyme

In some embodiments, the compound that down regulates the activity or expression level of PLD3 includes a ribosome that inhibits PLD3 protein expression.

A ribozyme is used to inhibit PLD3 protein expression. Ribozymes useful for inhibiting the expression of a target molecule may be designed by incorporating target sequences into the basic ribozyme structure which are complementary, for example, to the mRNA sequence encoding PLD3. Ribozymes are antisense RNAs which have a catalytic site capable of specifically cleaving complementary RNAs. Therefore, ribozymes having sequence complementary to PLD3 mRNA sequences are capable of downregulating the expression of PLD3 by reduces the level of PLD3 mRNA. Ribozymes targeting PLD3, may be synthesized using commercially available reagents (Applied Biosystems, Inc., Foster City, CA) or they may be genetically expressed from DNA encoding them. In some embodiments, the DNA encoding the ribozymes are incorporated in a vector, which is described in the “Vector” section elsewhere in the instant specification.

### Downregulating PLD3 by CRISPR Knockout/Knockdown and Other Knockouts/Knockdown Techniques

In some embodiments, the compound that down regulates the activity or expression level of PLD3 comprises a nucleic acid that down regulates the expression level of PLD3 by the means of CRISPR knockout.

In some embodiments, the compound down regulates the activity or expression level of PLD3 comprises a CRISPR/Cas9 system for knocking out PLD3.

The CRISPR/Cas9 system is a facile and efficient system for inducing targeted genetic alterations. Target recognition by the Cas9 protein requires a “seed” sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 293T cells), primary cells, and CAR T cells. The CRISPR/Cas9 system can simultaneously target multiple genomic loci by co-expressing a single Cas9 protein with two or more gRNAs, making this system uniquely suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The RecI domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US2014/0068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, an pAd5F35-CRISPR vector. In other embodiments, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

In certain embodiments, guide RNA(s) and Cas9 can be delivered to a cell as a ribonucleoprotein (RNP) complex. RNPs are comprised of purified Cas9 protein complexed with gRNA and are well known in the art to be efficiently delivered to multiple types of cells, including but not limited to neurons, stem cells and immune cells (Addgene, Cambridge, MA, Mirus Bio LLC, Madison, WI).

The guide RNA is specific for a genomic region of interest and targets that region for Cas endonuclease-induced double strand breaks. The target sequence of the guide RNA sequence may be within a loci of a gene or within a non-coding region of the genome. In certain embodiments, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

Guide RNA (gRNA), also referred to as “short guide RNA” or “sgRNA”, provides both targeting specificity and scaffolding/binding ability for the Cas9 nuclease. The gRNA can be a synthetic RNA composed of a targeting sequence and scaffold sequence derived from endogenous bacterial crRNA and tracrRNA. gRNA is used to target Cas9 to a specific genomic locus in genome engineering experiments. Guide RNAs can be designed using standard tools well known in the art.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In certain embodiments, the CRISPR/Cas is derived from a type II CRISPR/Cas system. In other embodiments, the CRISPR/Cas system is derived from a Cas9 protein. The Cas9 protein can be from Streptococcus pyogenes, Streptococcus thermophilus, or other species.

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the instant specification. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the instant specification may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4^{th} Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

In some embodiments, the compound that down regulates the activity or expression level of PLD3 comprises a nucleic acid that down regulates the expression level of PLD3 by the means of CRISPR knockdown. CRISPR knockdown includes, but not limited to, CRISPRCas13 knockdown. (See e.g., Mendez-Mancilla et al., Cell Chemical Biology 29, 1-7, 2021 Jul. 27, and Kushawah et al., Dev Cell. 2020 Sep. 28; 54 (6): 805-817. The entireties of which are incorporated herein by reference).

In some embodiments, the present invention includes any other methods for effecting gene knockdown and/editing, which allow for deletion and/or inactivation of PDL3, such as but not limited to those described in WO 2018/236840 (which is incorporated herein in its entirety by reference).

Downregulating PLD3 by Inactivating and/or Sequestering

In some embodiments, the compound that downregulates the activity or expression level of PLD3 includes a protein that downregulates the activity of PLD3 by inactivating and/or sequestering PDL3. In some embodiment, the compound includes a nucleic acid that express the protein that downregulates the activity of PLD3 by inactivating and/or sequestering PDL3. In some embodiments, the compound includes an expression vector that express the protein that downregulates the activity of PLD3 by inactivating and/or sequestering PDL3 (see “Vector” section for descriptions on vectors).

In some embodiments, the compound that downregulates the expression level of PLD3 is a trans-dominant negative mutant of PLD3, and/or a nucleic acid or a vector expressing the trans-dominant negative mutant of PLD3.

Method of Treating, Ameliorating, and/or Preventing Neurodegenerative Condition

The formation of large axonal enlargements (i.e., the spheroids) is a neurological condition commonly found in many types of neurodegenerative conditions other than Alzheimer's disease. For example, Lou Gehrig's disease (ALS), Huntington's disease, post traumatic encephalopathy, lysosomal storage disorders including Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia are all known to involve axonal spheroids.

As detailed elsewhere in the instant specification, the present study demonstrated that the formation and enlargement of axonal spheroids disrupt the propagation of action potentials (APs) along the axons. Using Alzheimer's disease as a model, the present study demonstrated that axonal spheroids associated with amyloid plaques (also referred to as “plaque-associated axonal spheroids” or “PAAS” herein) disrupt the propagation of action potentials by acting as electric current sinks. the present study demonstrated methods of reducing the sizes of the axonal spheroids, and that doing so restores the axonal conduction properties of the axons affected by the axonal spheroids.

As detailed elsewhere in the instant specification, the present study demonstrated that downregulating PLD3 expression level reverses the enlargement of PAAS and restores the axonal conduction properties of the axons affected by the axonal spheroids, while overexpression of PLD3 has the opposite effects.

Therefore, in some embodiments, the instant specification is directed to a method of treating ameliorating, and/or preventing a neurodegenerative condition in a subject. In some embodiments, the neurodegenerative disease is Alzheimer's disease, Lou Gehrig's disease (ALS), Huntington's disease, post traumatic encephalopathy, lysosomal storage disorders including Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, Lewy Body dementia, or combinations thereof. In some embodiments, the neurodegenerative disease is Alzheimer's disease. In some embodiments, the subject is a mammal. In some embodiments, the mammal is a human.

In some embodiments, the method reverses, ameliorates, and/or prevents the formation or enlargement of axonal spheroids in the subject. In some embodiments, the method reverses or prevents the formation and/or enlargement of axonal spheroids associated with amyloid plaques found in an Alzheimer's disease patient. In some embodiments, the method restores a propagation of an action potential (AP) along an axon of a neuron blocked or delayed by an axonal spheroid on the axon.

In some embodiments, the method of treating, ameliorates, and/or preventing a neurodegenerative condition in a subject includes administering to a subject in need thereof a compound that downregulates an activity and/or expression level of PLD3 in a neuron affected by the neurodegenerative condition. In some embodiments, administering to the subject in need thereof a compound that downregulates the activity and/or expression level of PLD3 includes administering to the subject in need thereof an effective amount of the compound that downregulates the activity and/or expression level of PLD3. What is considered as “effective amount” by the specification is described elsewhere herein.

In some embodiments, the compound that downregulates the expression level and/or activity of PLD3 includes a small molecule inhibitor of PLD3, a protein inhibitor of PLD3, or a compound that downregulates the expression level or the activity of PLD3 by RNA interference, by ribozyme, by CRISPR knockout/knockdown, or by producing a trans-dominant negative mutant, and so forth. The exemplary compounds described in this paragraph are the same as or similar to those as described above in the “Method of Reversing or Preventing Formation or Enlargement of Axonal Spheroids” section.

### Composition for Treating Neurodegenerative Condition

The formation of large axonal enlargements (i.e., the spheroids) is a neurological condition commonly found in many types of neurodegenerative conditions other than Alzheimer's disease. For example, Lou Gehrig's disease (ALS), Huntington's disease, post traumatic encephalopathy, lysosomal storage disorders including Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia are all known to involve axonal spheroids.

As detailed elsewhere in the instant specification, the present study demonstrated that the formation and enlargement of axonal spheroids disrupt the propagation of action potentials (APs) along the axons. Using Alzheimer's disease as a model, the present study demonstrated that axonal spheroids associated with amyloid plaques (also referred to as “plaque-associated axonal spheroids” or “PAAS” herein) disrupt the propagation of action potentials by acting as electric current sinks. The present study further demonstrated methods of reducing the sizes of the axonal spheroids, and that doing so restores the axonal conduction properties of the axons affected by the axonal spheroids.

As detailed elsewhere in the instant specification, the present study demonstrated that compounds that downregulate PLD3 expression level can reverse the enlargement of PAAS and restore the axonal conduction properties of the axons affected by the axonal spheroids.

Therefore, in some embodiments, the instant specification is directed to a composition for treating a neurodegenerative condition in a subject. In some embodiments, the composition includes a compound that downregulates an expression level and/or activity of PLD3 in a neuron affected by an axonal spheroid, and at least one pharmaceutically acceptable carrier. In some embodiments, the compound that downregulates the expression level and/or activity of PLD3 in the neuron stops and/or reverses the enlargement of the axonal spheroid.

In some embodiments, the compound that downregulates an expression level or an activity of PLD3 includes a small molecule inhibitor of PLD3, a protein inhibitor of PLD3, a nucleic acid that causes RNA interference on the expression of PLD3, a ribozyme that inhibits the expression of PLD3, a CRISPR system that knocks out or knocks down PLD3, a trans-dominant negative mutant of PLD3, and so forth. The exemplary compounds described in this paragraph are the same as or similar to those as described above in the “Method of Reversing or Preventing Formation or Enlargement of Axonal Spheroids” section.

### Vectors

Vectors can increase the stability of the nucleic acids, make the delivery easier, or allow the expression of the nucleic acids or protein products thereof in the cells.

Therefore, in some embodiments, the protein inhibitors or the nucleic acids that that down regulates the activity or expression level of PLD3 is incorporated into a vector.

In some embodiments, the instant specification relates to a vector, including the nucleic acid sequence of the instant specification or the construct of the instant specification. The choice of the vector will depend on the host cell in which it is to be subsequently introduced. In certain embodiments, the vector of the instant specification is an expression vector. Suitable host cells include a wide variety of prokaryotic and eukaryotic host cells. In certain embodiments, the expression vector is selected from the group consisting of a viral vector, a bacterial vector and a mammalian cell vector. Prokaryote- and/or eukaryote-vector based systems can be employed for use with the instant specification to produce polynucleotide, or their cognate polypeptides. Many such systems are commercially and widely available.

In some embodiments, the vector is a viral vector. Viral vector technology is well known in the art and is described, for example, in virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

In some embodiments, the viral vector is a suitable adeno-associated virus (AAV), such as the AAV1-AAV8 family of adeno-associated viruses. In some embodiments, the viral vector is a viral vector that can infect a human. The desired nucleic acid sequence, such as the nucleic acids that downregulates PLD3 described above, can be inserted between the inverted terminal repeats (ITRs) in the AAV. In various embodiments, the viral vector is an AAV2 or an AAV8. The promoter can be a thyroxine binding globulin (TBG) promoter. In various embodiments, the promoter is a human promoter sequence that enables the desired nucleic acid expression in the brain. In some embodiments, the promoter is a neuron-selective promoter or a neuron-specific promoter. The AAV can be a recombinant AAV, in which the capsid comes from one AAV serotype and the ITRs come from another AAV serotype. In various embodiments, the AAV capsid is selected from the group consisting of a AAV1, AAV2, AAV3, AAV4, AAV5, AAV6, AAV7, and a AAV8 capsid. In various embodiments, the ITR in the AAV is at least one ITR selected from the group consisting of a AAV1, AAV2, AAV3, AAV4, AAV5, AAV6, AAV7, and an AAV8 ITR. In various embodiments, the instant specification contemplates an AAV8 viral vector (recombinant or non-recombinant) containing a desired nucleic acid expression sequence and at least one promoter sequence that, when administered to a subject, causes elevated systemic expression of the desired nucleic acid. In some embodiments, the viral vector is a recombinant or non-recombinant AAV2 or AAV5 containing any of the desired nucleic acid expression sequences described herein. In some embodiments, the AAV is an engineered AAVs for delivering nucleic acid across the blood brain barrier to the central and peripheral nervous systems, such as those as described by Chan et al., Nat Neurosci. 2017 August; 20 (8): 1172-1179. The entirety of this reference is incorporated herein by reference.

In some embodiments, the vector in which the nucleic acid sequence is introduced is a plasmid that is or is not integrated in the genome of a host cell when it is introduced in the cell. Illustrative, non-limiting examples of vectors in which the nucleotide sequence of the instant specification or the gene construct of the instant specification can be inserted include a tet-on inducible vector for expression in eukaryote cells.

The vector may be obtained by conventional methods known by persons skilled in the art (Sambrook et al., 2012). In certain embodiments, the vector is a vector useful for transforming animal cells.

In certain embodiments, the recombinant expression vectors may also contain nucleic acid molecules which encode a peptide or peptidomimetic inhibitor of the instant specification, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

It will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression. The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

### Combination Therapies

In some embodiments, the method of treating, ameliorating, and/or preventing the neurodegenerative condition or the method of reversing or preventing formation and/or enlargement of axonal spheroids includes administering to the subject the effective amount of at least one compound and/or composition contemplated within the disclosure.

In some embodiments, the composition for treating neurodegenerative condition includes at least one compound and/or composition contemplated within the disclosure.

In some embodiments, the subject is further administered at least one additional agent that treats, ameliorates, and/or prevents a disease and/or disorder contemplated herein. In other embodiments, the compound and the at least one additional agent are co-administered to the subject. In yet other embodiments, the compound and the at least one additional agent are co-formulated.

The compounds contemplated within the disclosure are intended to be useful in combination with one or more additional compounds. These additional compounds may comprise compounds of the present disclosure and/or at least one additional agent for treating neurodegenerative conditions, and/or at least one additional agent that treats one or more diseases or disorders contemplated herein.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations contemplated within the disclosure may be administered to the subject either prior to or after the onset of a disease and/or disorder contemplated herein. Further, several divided dosages, as well as staggered dosages may be administered daily or sequentially, or the dose may be continuously infused, or may be a bolus injection. Further, the dosages of the therapeutic formulations contemplated within the disclosure may be proportionally increased or decreased as indicated by the exigencies of the therapeutic or prophylactic situation.

Administration of the compositions contemplated within the disclosure to a patient, preferably a mammal, more preferably a human, may be carried out using known procedures, at dosages and for periods of time effective to treat a disease and/or disorder contemplated herein in the patient. An effective amount of the therapeutic compound necessary to achieve a therapeutic effect may vary according to factors such as the state of the disease or disorder in the patient; the age, sex, and weight of the patient; and the ability of the therapeutic compound contemplated within the disclosure to treat a disease and/or disorder contemplated herein in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound contemplated within the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions contemplated within the disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds contemplated within the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms contemplated within the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease and/or disorder contemplated herein.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the disclosure are administered to the patient in dosages that range from one to five times per day or more. In another embodiment, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of neurodegenerative conditions in a patient.

Formulations may be employed in admixtures with conventional excipients, i.e., pharmaceutically acceptable organic or inorganic carrier substances suitable for intracranially, intrathecal, oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

The present disclosure also includes a multi-layer tablet comprising a layer providing for the delayed release of one or more compounds of the disclosure, and a further layer providing for the immediate release of another medication. Using a wax/pH-sensitive polymer mix, a gastric insoluble composition may be obtained in which the active ingredient is entrapped, ensuring its delayed release.

### Parenteral Administration

For parenteral administration, the compounds of the disclosure may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. Patents Applications Nos. 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure depends on the age, sex and weight of the patient, the current medical condition of the patient and the progression of the neurodegenerative condition in the patient being treated. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the modulator of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the patient's condition, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. Capsid assembly modulators exhibiting high therapeutic indices are preferred. The data obtained from cell culture assays and animal studies are optionally used in formulating a range of dosage for use in human. The dosage of such capsid assembly modulators lies preferably within a range of circulating concentrations that include the ED₅₀ with minimal toxicity. The dosage optionally varies within this range depending upon the dosage form employed and the route of administration utilized.

Those skilled in the art recognizes, or is able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in assay and/or reaction conditions, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: Functional Consequences of Axonal Spheroids

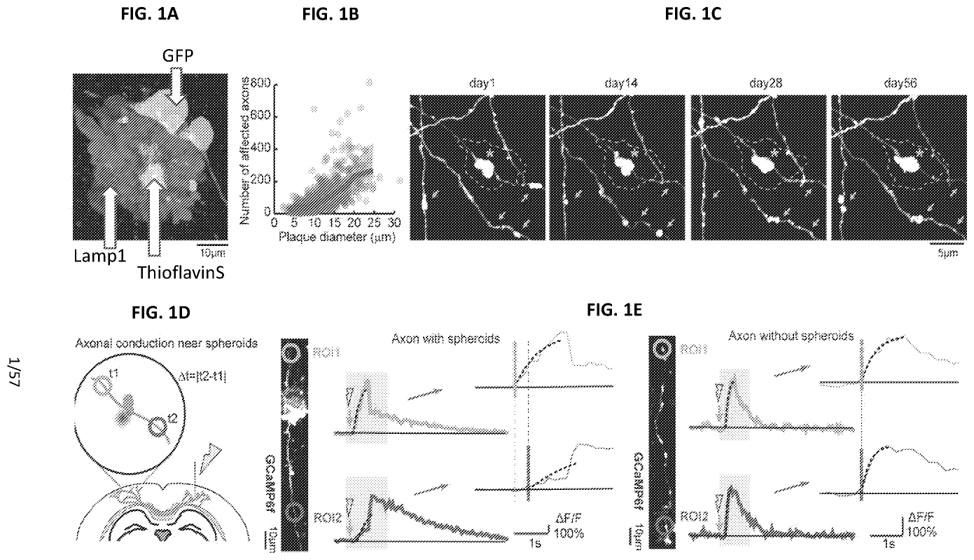

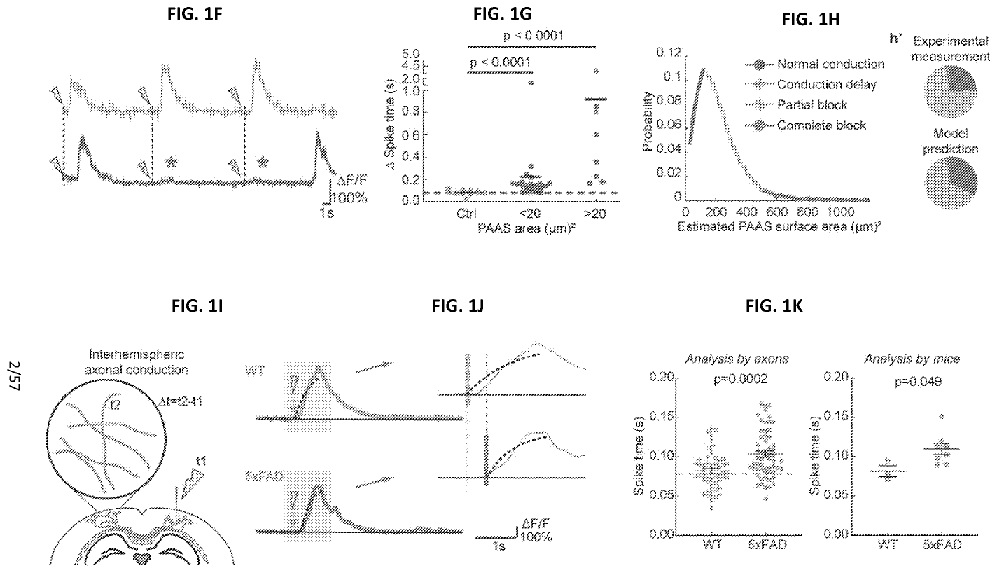

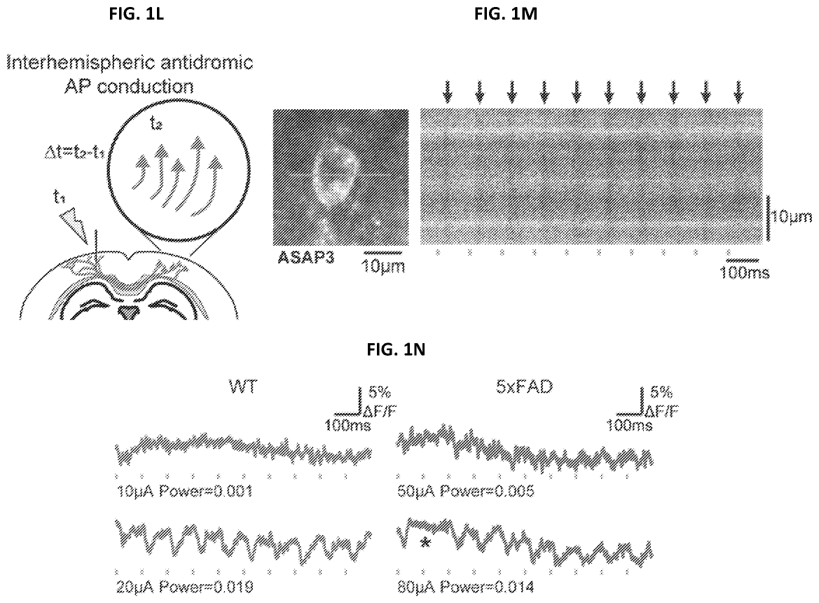

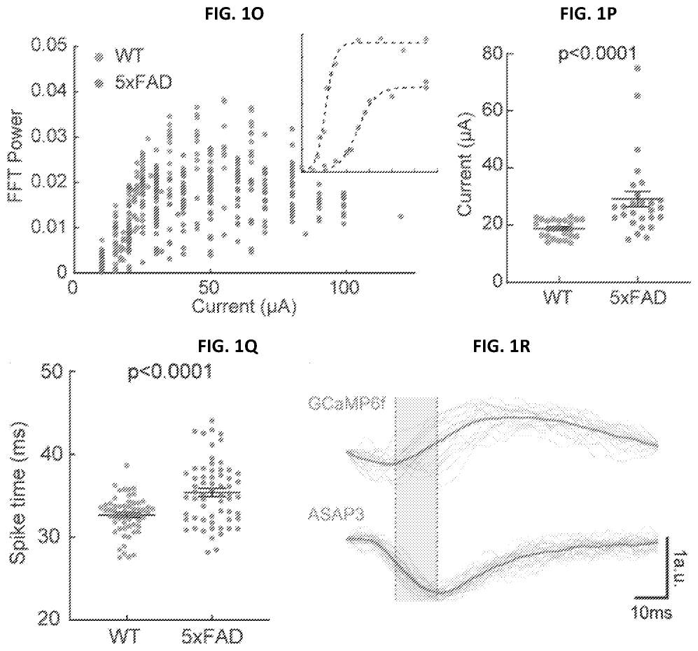

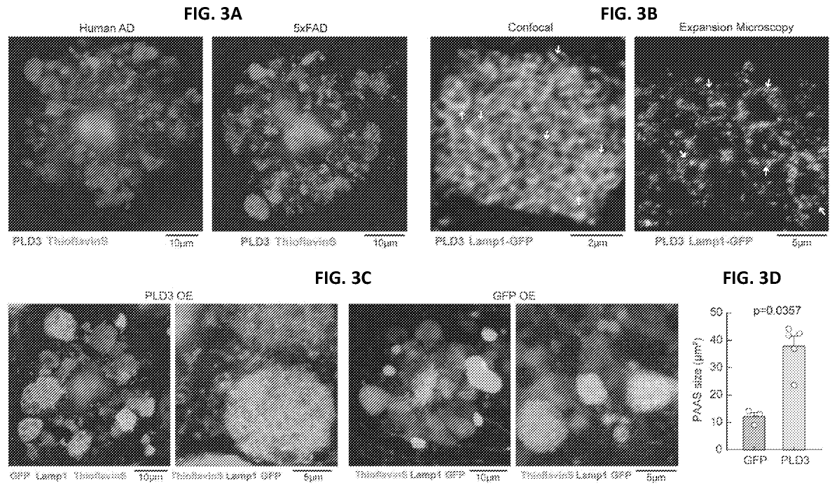

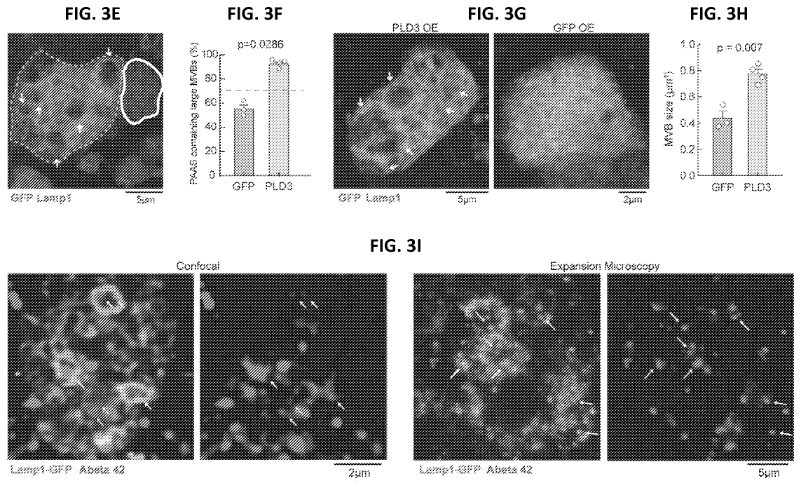

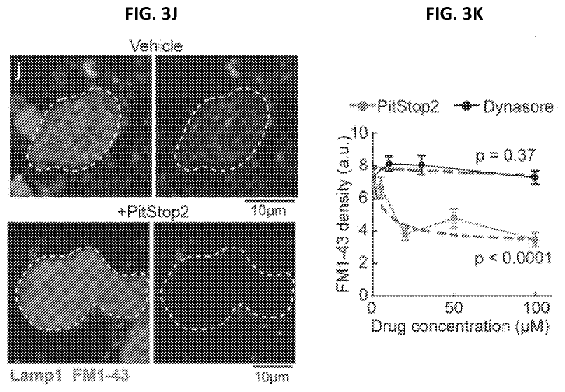

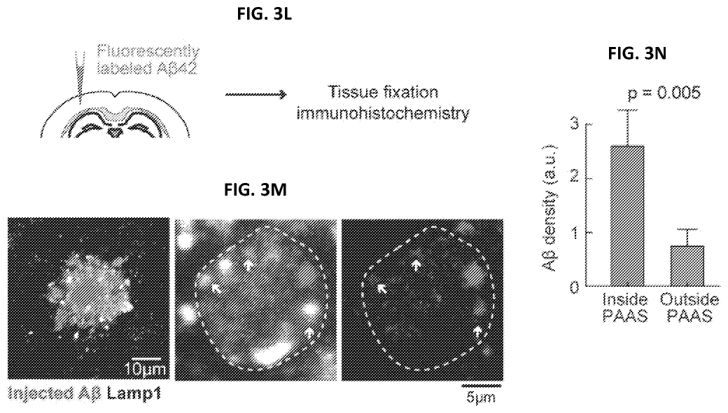

Axonal spheroids are found abundantly around individual amyloid plaques in both AD-like mice and human AD patients (FIGS. 1A and 3A). Based on the average volume of individual PAAS and the total volume of the PAAS halo around plaques, the present study demonstrated that individual plaques can on average affect hundreds of axons (FIG. 1B, also discussed in Example 9). Given the abundance of amyloid plaques in the AD brain, this suggests that massive numbers of axons can be affected, highlighting the potential significance of PAAS as a mechanism of neural network dysfunction. Time lapse imaging of virally labeled axons around plaques revealed that PAAS can be very stable over intervals of up to months (FIGS. 1C and 5C-5E). While most PAAS increased in size overtime, a substantial number declined in volume or disappeared during this interval, without the loss of the parent axon (FIGS. 1C and 5C-5E). This indicates that PAAS are not a feature of degenerating axons but are instead stable axonal structures that may affect neuronal circuits for extended intervals, while at the same time having the potential for reversibility.

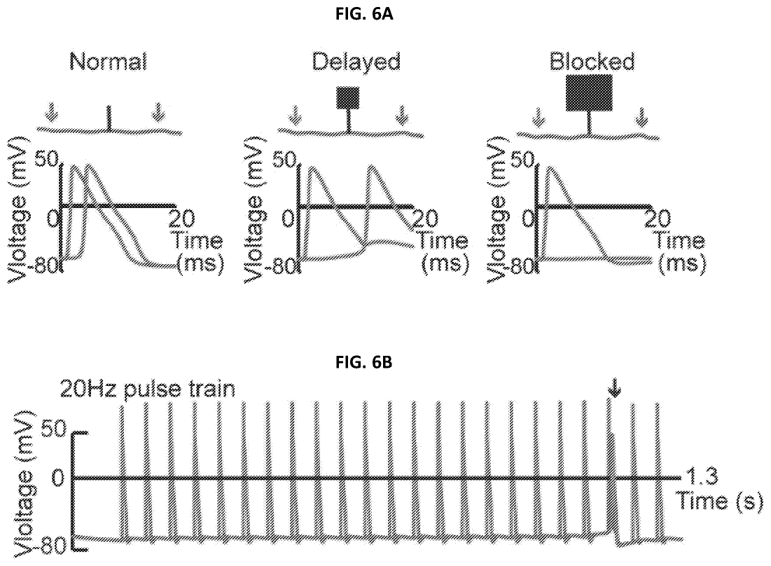

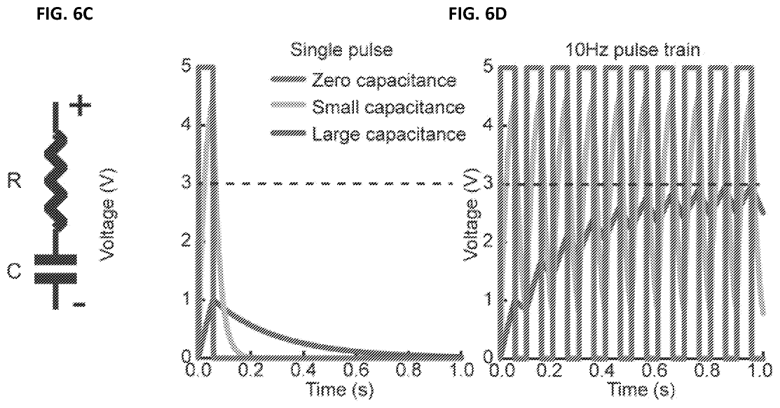

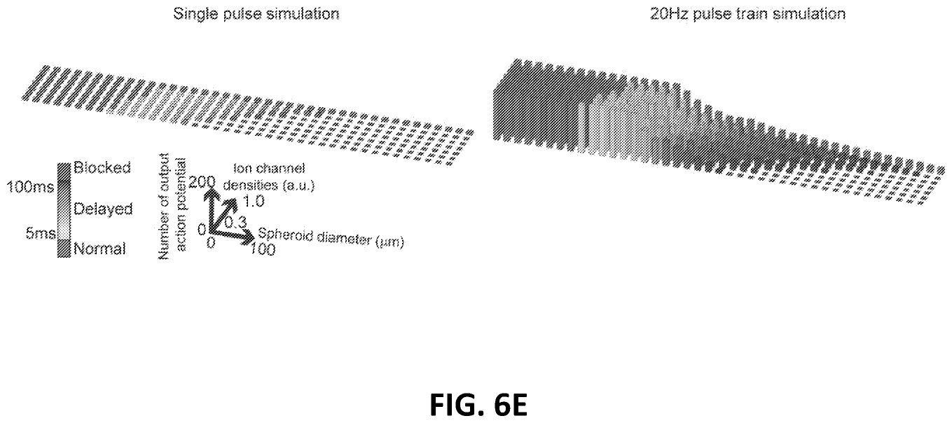

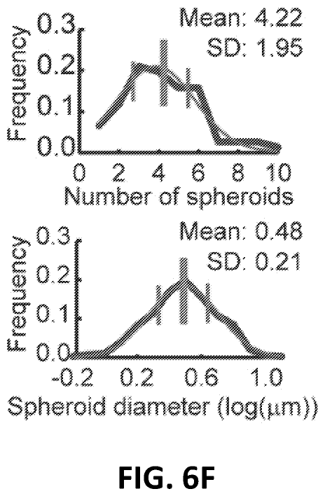

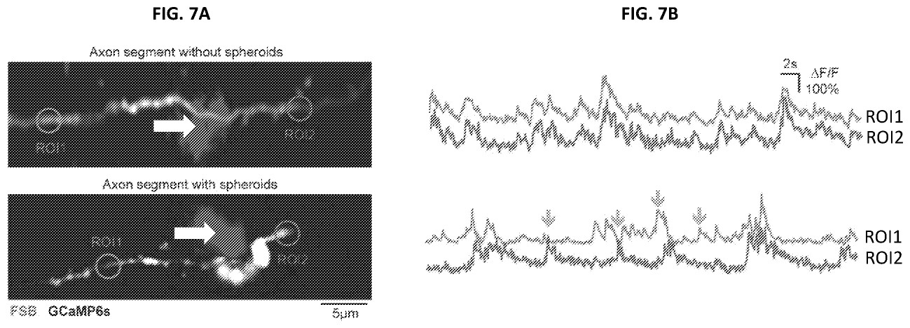

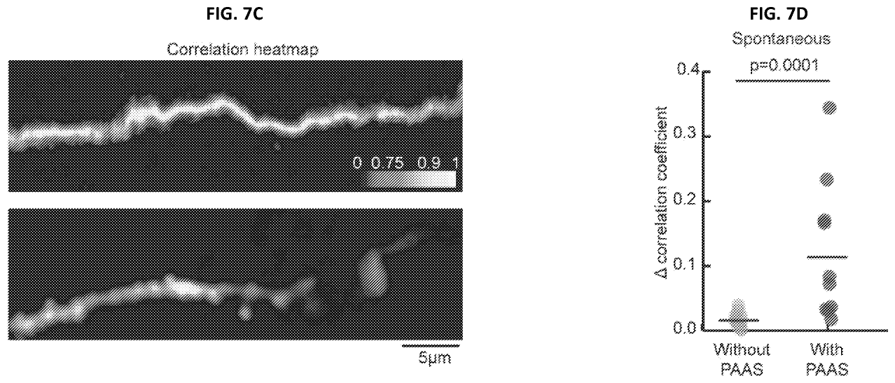

To examine how PAAS might disrupt neuronal circuits, the present study computationally modeled how these spheroids affect axonal conduction. It was found that the likelihood of conduction disruption of action potentials (AP) for a particular axonal segment, markedly increased as a function of the total PAAS surface area (FIGS. 6A-6F). The in silico model predicted that PAAS behave as capacitors that act as electric current sinks for incoming action potentials and can thus cause conduction blocks or prolonged delays (FIGS. 6A-6F, also discussed in Example 10). To experimentally examine the electrical conduction properties of axons, a strategy for measuring the propagation of action potentials in individual axons was developed through Ca²⁺ imaging in the live mouse brain. The calcium sensor, GCaMP6f, was virally expressed through delivery of adeno-associated viral (AAV) vectors to one hemisphere of the mouse brain (FIG. 5A) and performed Ca²⁺ imaging of individual projection axons on the contralateral cortex (FIG. 1D). AP propagation was measured after electrical stimulation of the ipsilateral hemisphere with trains of electrical pulses and the rise times of Ca²⁺ transients (a surrogate for AP spike time) was compared at two regions of interest (ROI) located on axon segments on both sides of individual PAAS (FIG. 1D). The onset of the rise-times was consistently delayed over intervals ranging from hundreds of milliseconds to seconds (FIGS. 1E-1G). Given that the present study used a series of pulses of electrical stimulation to induce trains of AP spikes, it was concluded that the unusually long delays in Ca²⁺ rise-times observed, were due to conduction blocks of a substantial proportion of individual AP spikes, once they reached individual spheroids (FIG. 6B). On the other hand, comparison of rise times at two ROIs in axon segments without spheroids or at ROIs located on the same side of axonal segments adjacent to spheroids (FIG. 1E, right panel) demonstrated no difference in the onset of rise-times, strongly suggesting a deleterious effect of PAAS, rather than broad spheroid-independent defects in axonal conduction. Furthermore, in addition to electrically evoked responses, spontaneous Ca²⁺ transients were also imaged in individual axons and similar conduction abnormalities were observed in segments with PAAS (FIGS. 7A-7D). Importantly, regardless of whether the APs were the result of spontaneous or electrically induced neuronal activity, larger spheroids were observed to cause more severe conduction blocks (FIGS. 1G and 7D). These experimental observations were consistent with the computational modeling demonstrating that the size of individual PAAS is a determinant of the degree of conduction defects associated with spheroids (FIGS. 1H and 6A-6F).

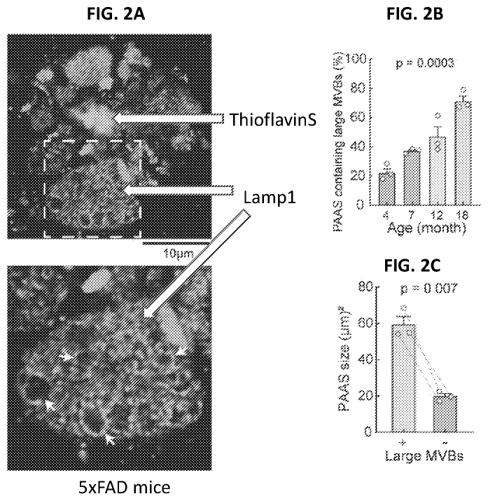

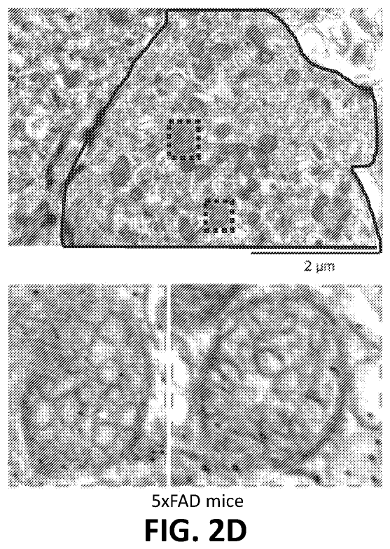

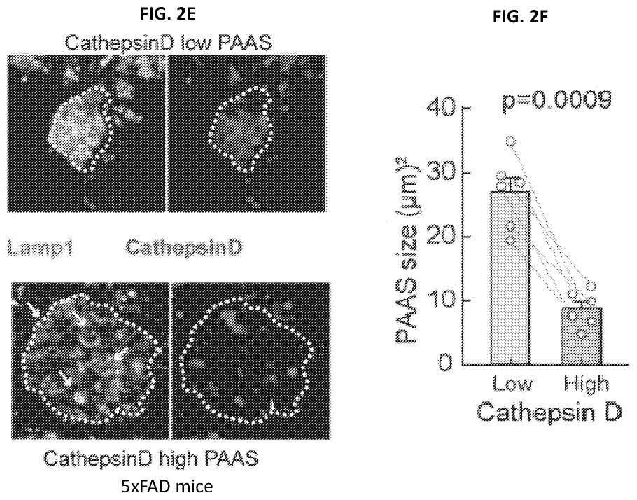

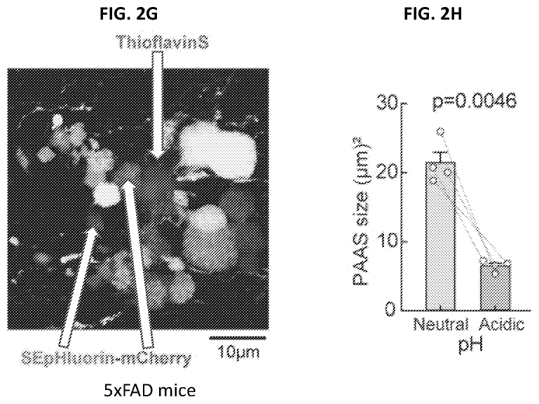

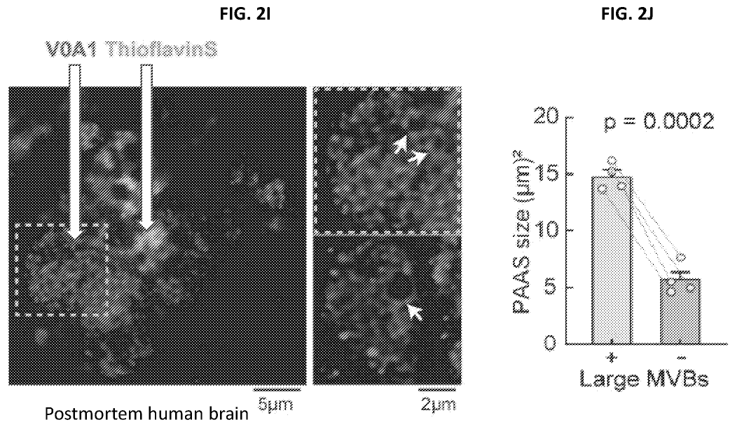

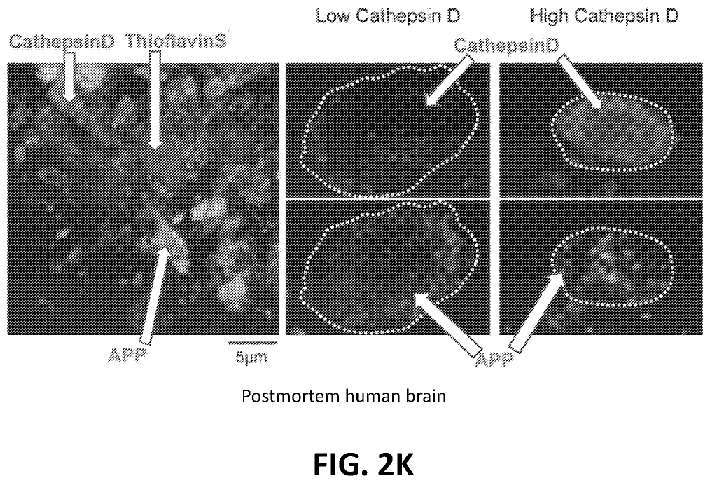

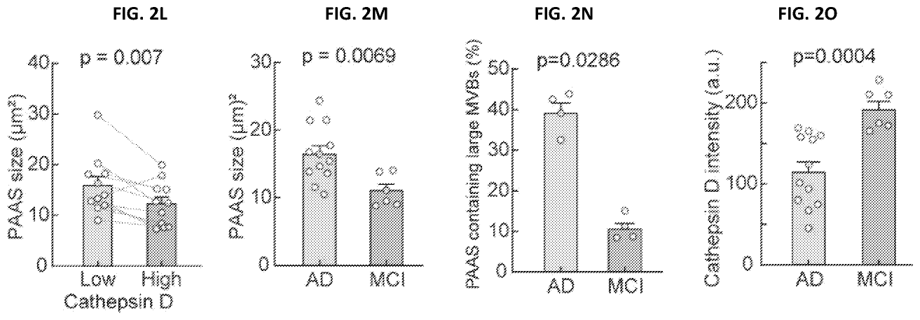

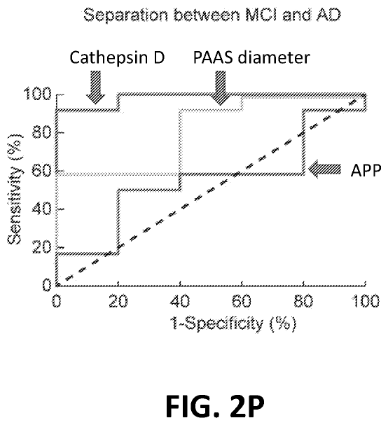

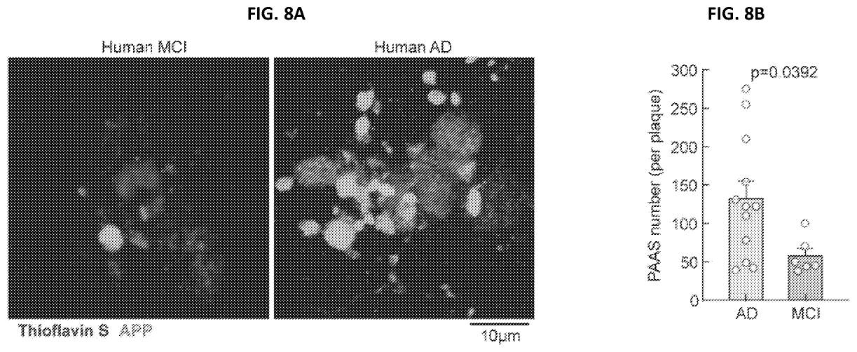

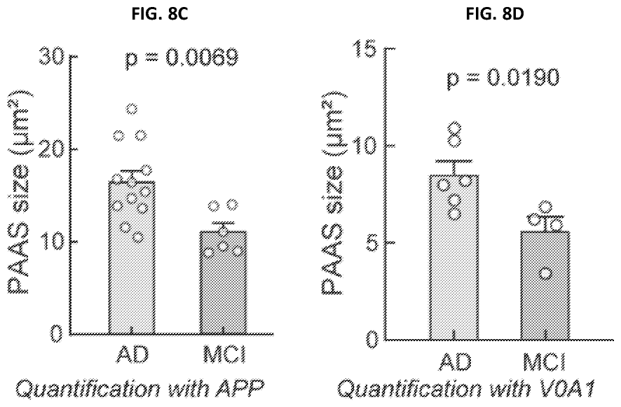

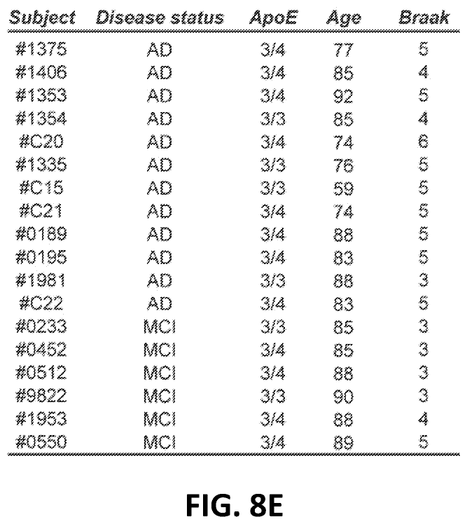

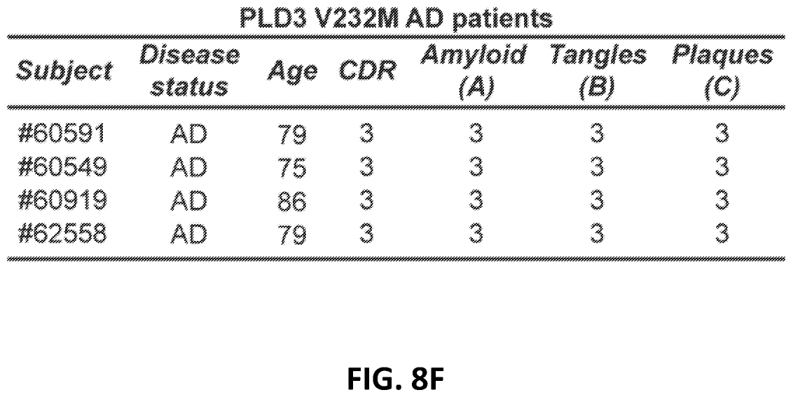

Given that the present study found marked abnormalities in local axonal conduction around plaques, it was further explored if this was associated with more widespread defects in long-range cortical connectivity, by measuring interhemispheric conduction velocity through calcium imaging in live 5×FAD mice. To achieve this, AAV9-Syn-GCaMP6f was stereotaxically injected to label a homogeneous population of closely located cortical neurons in somatosensory cortex, which assured comparable axonal distances to the contralateral hemisphere imaging region across different mice. Contralateral projecting axons were then imaged, while electrically stimulating the ipsilateral GCaMP6f-labeled neurons (FIG. 1I). It was found that the interhemispheric conduction velocities in wildtype mice were of similar magnitude as those previously reported using slice electrophysiology recordings. However, in 5×FAD mice, it was found that Ca²⁺ rise times in projecting axons were markedly delayed (FIGS. 1J-1K). This suggests that given the long interhemispheric distances, the probability of axons to encounter amyloid plaques and develop spheroids is relatively high. While the density of amyloid plaques in humans is lower than in mice, the axonal lengths are much greater, thereby increasing the probability of adjacency to amyloid plaques, resulting in potential disruption of axonal connectivity. Importantly, through quantitative analysis of human postmortem brains, a much greater average number of axonal spheroids per amyloid plaque (FIGS. 8A-8B) and greater PAAS size (FIG. 2M) was found in individuals with moderate to severe AD, compared to those with mild cognitive impairment (MCI). Altogether, this suggests that both PAAS number and size could be important factors that determine the degree of neural circuit disruption and cognitive deficits in AD.

### Example 2: Disruption of Long-Range Axonal Connectivity

Given that the present study found marked abnormalities in local axonal conduction around plaques, if this was associated with more widespread defects in long-range cortical connectivity was further explored. The present study thus developed a strategy for measuring interhemispheric conduction velocity through calcium imaging in live 5×FAD mice. To achieve this, AAV9-Syn-GCaMP6f was stereotaxically injected to label a homogeneous population of closely located cortical neurons in somatosensory cortex, which assured comparable axonal distances to the imaged regions on the contralateral hemisphere across different mice. The present study then imaged contralateral projecting axons, while electrically stimulating the ipsilateral GCaMP6f-labeled neurons (FIG. 1I). It was found that the interhemispheric conduction velocities in wildtype mice were of similar magnitude as those using slice electrophysiology recordings. However, in 5×FAD mice, it was found that Ca²⁺ rise times in projecting axons were markedly delayed (FIGS. 1J-1K). This suggests that local action potential conduction abnormalities caused by PAAS lead to disruption in long-range functional axonal connectivity. To further validate that the observations, as measured by calcium imaging, reflect actual action potential delays or blockades, the present study implemented in vivo voltage imaging using the genetically encoded voltage sensor ASAP325. AAV2-Syn-ASAP3 was intra-cortically injected in the same manner as was done for GCaMP6f (FIG. 1L). Given the relatively low signal-to-noise ratio when imaging with genetically encoded voltage sensors, it was not feasible to perform single-trial experiments of individual axons. Instead, the present study stimulated the axons in one hemisphere and recorded the contralateral hemisphere antidromic action potential, visualized at the cell bodies rather than axons (FIGS. 1L-1M), which markedly improved signal-to-noise ratio. Using this strategy, it was found that in 5×FAD mice, there was a marked increase in the electric current required to induce the interhemispheric propagation of an action potential through single trial stimulations (FIGS. 1N-1P), consistent with PAAS acting as current sinks. Furthermore, the present study also observed frequent delays on AP propagation when comparing 5×FAD and WT mice (FIGS. 1Q-1R) in agreement with the Ca²⁺ imaging experiments. Altogether, the imaging data as well as the computational modeling highlight the prevalence of action potential conduction blocks resulting from spheroid pathology in AD. Given the long interhemispheric distances, the probability of axons to encounter amyloid plaques and develop spheroids is relatively high. While the density of amyloid plaques in humans is lower than in mice, the present study hypothesizes that given their much greater axonal lengths in humans, this would increase the probability of adjacency to amyloid plaques, and thus the likelihood of disruption in axonal connectivity. In support of this idea, through quantitative analysis of postmortem brains, the present study found a much greater average number of axonal spheroids per amyloid plaque and greater PAAS size (FIGS. 8A-8C) in individuals with moderate to severe AD, compared to those with mild cognitive impairment (MCI). This suggests that both PAAS number and size could be important factors that determine the degree of neural circuit disruption and cognitive deficits in AD.

### Example 3: Multivesicular Bodies Drive PAAS Enlargement

High-resolution confocal imaging revealed that as mice aged, there was a progressive accumulation of aberrantly enlarged LAMP-1 (lysosome associated membrane protein 1)-positive vesicles within PAAS (FIGS. 2A-2B). In addition, there was a striking correlation between the presence of enlarged LAMP-1 positive vesicles and the overall size of individual PAAS (FIG. 2C). Electron microscopy (EM) imaging revealed that these large vesicles were densely filled with smaller intraluminal vesicles (FIG. 2D), consistent with the ultrastructural characteristics of multivesicular bodies (MVBs), which are considered precursors of mature lysosomes. Small PAAS were found to be predominantly filled with vesicles that contained high levels of the protease cathepsin D and were acidic (as measured with the pH-sensitive genetically-encoded reporter SEpHluorin), which are characteristics of lysosomes (FIGS. 2E-2H). However, as PAAS enlarged, the overall acidification and cathepsin D levels in individual PAAS declined (FIGS. 2E-2H), coinciding with the accumulation of MVBs, which typically have not yet acquired lysosomal proteases and acidic pH. This suggests that spheroid enlargement is mechanistically linked with the accumulation of enlarged MVBs in mice. Similar MVB-like structures were observed within spheroids surrounding amyloid plaques in postmortem human brains (FIG. 2I), and their presence was associated with larger spheroid size (FIG. 2J). In accordance with the abundance of immature MVBs in humans, larger PAAS contained lower levels of cathepsin D (FIGS. 2K-2L). Importantly, consistent with the observation that PAAS size is inversely correlated with premortem cognitive function (FIG. 2M), a similar correlation between premortem cognition, the abundance of large MVBs and low levels of cathepsin D was found within PAAS (FIGS. 2N-2P). These data indicate in certain non-limiting embodiments that accumulation of MVBs drives the enlargement of axonal spheroids, which disrupts axonal conduction and ultimately affects cognition.

### Example 4: Role of PLD3 in Multivesicular Body Abnormalities

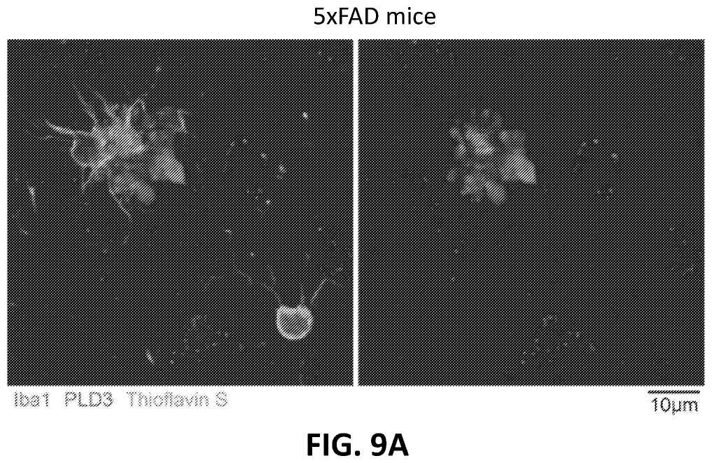

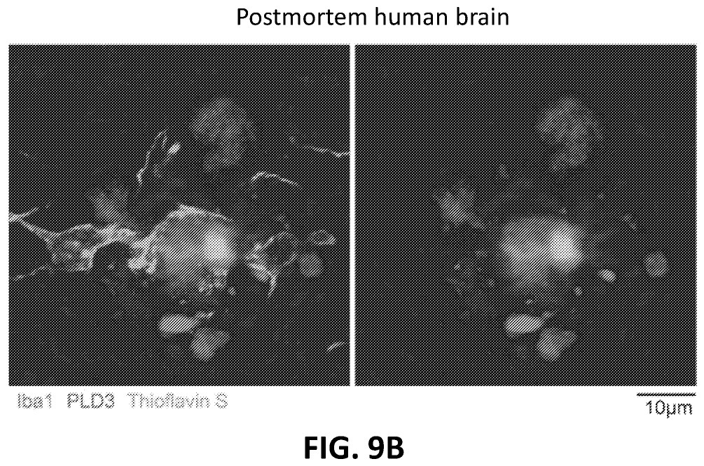

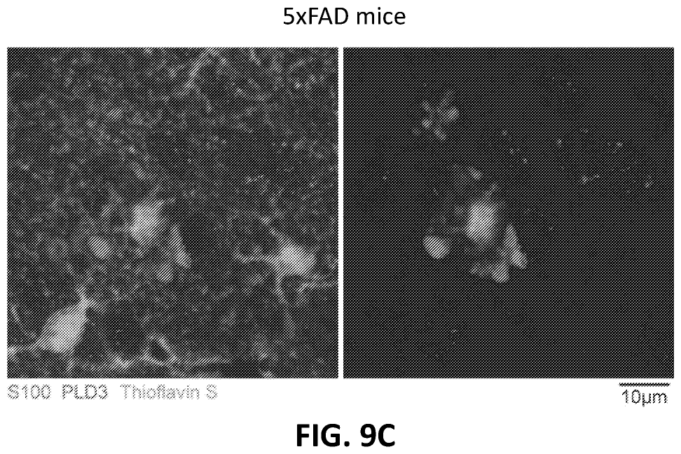

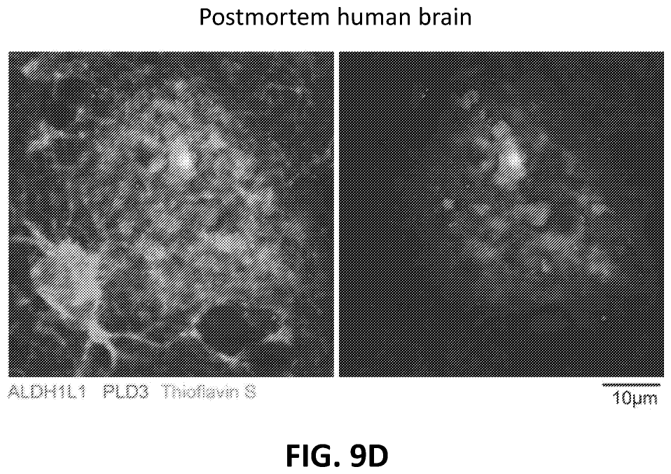

Potential mechanisms of accumulation of MVBs within axonal spheroids were then explored. PLD3 is a lysosomal protein that is of potential interest because it strongly accumulates in axonal spheroids in both humans and mice (FIG. 3A) and its expression is not detectable in other cell types such as microglia and astrocytes (FIGS. 9A-9D). In addition, PLD3 genetic variants may increase the risk of AD, although this remains a topic of controversy. Interestingly, PLD3 is considered to be the only lysosomal resident protein that is sorted into intraluminal vesicles (IL Vs) of MVBs in mammals, in contrast to the majority of lysosomal resident proteins which are sorted to the limiting membranes of MVBs. Indeed, immunofluorescence confocal imaging of axonal spheroids showed accumulation of PLD3 within the lumen of LAMP-1 positive vesicular structures (FIG. 3B, left panel), and expansion microscopy (ExM) revealed a punctate signal within MVBs, consistent with PLD3 positive ILVs (FIG. 3B, right panel), similar to previous immunogold electron microscopy of cultured cells. Together, these observations raise the possibility that PLD3 may play a role in the formation of MVBs, thereby affecting their accumulation and the consequent enlargement of axonal spheroids.

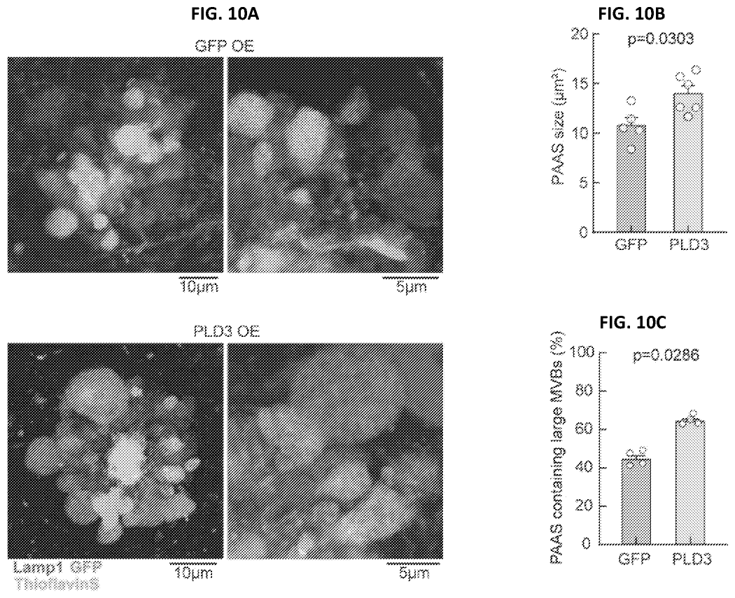

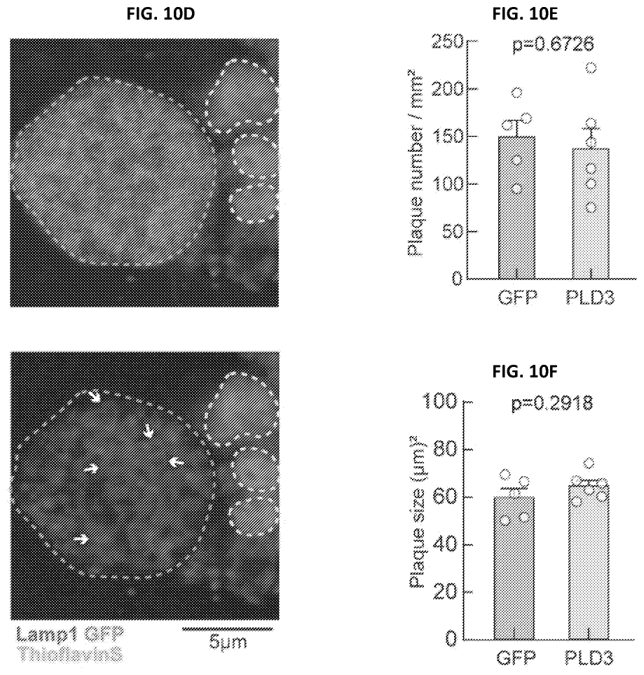

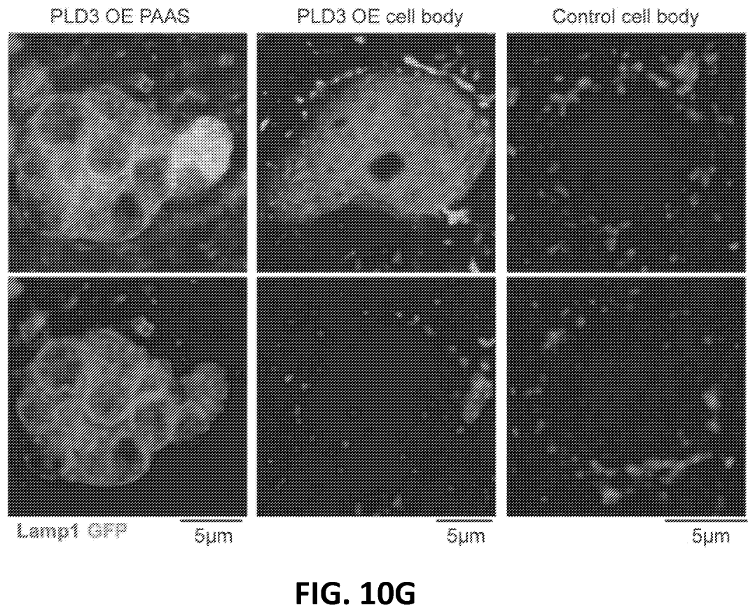

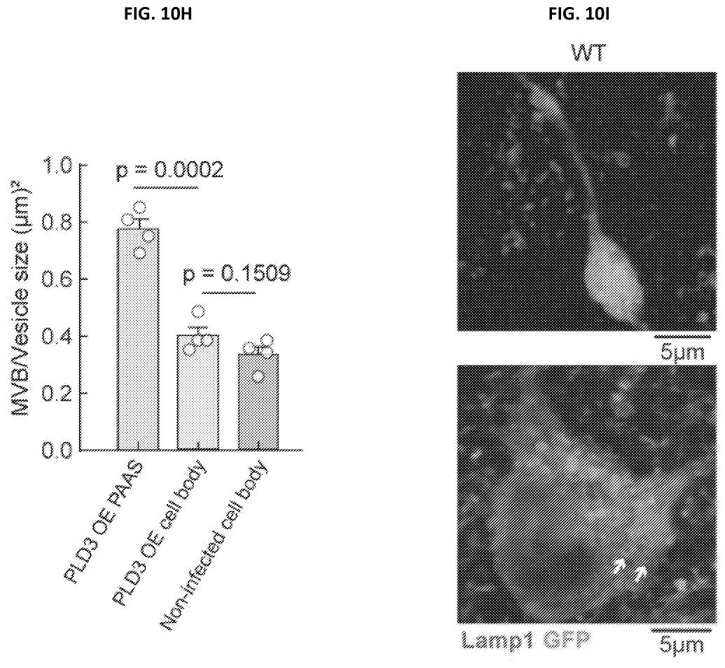

To further understand the role of PLD3 in the evolution of axonal spheroid pathology, in vivo AAV2-mediated overexpression of PLD3 was implemented in neurons of 5×FAD mice. It was observed that spheroids in PLD3 overexpressing axons were markedly larger than those expressing only GFP (Green Fluorescent Protein) (FIGS. 3C-3D and 10A-10B). Notably, confocal microscopy of individual spheroids revealed an increase in the number of large MVBs, even beyond what is seen in advanced aging in 5×FAD mice (FIGS. 3E-3F and 10C-10D). Furthermore, there was a marked increase in MVB size within PLD3 overexpressing spheroids compared to GFP-expressing controls (FIGS. 3G-3H). This manipulation was not associated with changes in amyloid plaque number or size (FIGS. 10E-10F), suggesting that PLD3 overexpression does not affect amyloid precursor protein (APP) processing. The enlargement and accumulation of large LAMP1-positive vesicles within PLD3 overexpressing spheroids, which are uniformly in the vicinity of amyloid plaques, was much greater than within cell bodies that were distant from plaques (FIGS. 1-G-10H). This suggests that Aβ from extracellular deposits is critical for PLD3-induced MVB abnormalities in axons. Indeed, using an antibody that specifically recognizes the Aβ-42 peptide, Aβ accumulation was observed within large MVBs (FIG. 3I), where it could potentially synergize with PLD3. The source of Aβ-42 within MVBs is likely to be endocytosis of oligomeric peptides from adjacent amyloid plaques. This is supported by the data showing that spheroids are sites of very active endocytosis (FIGS. 3J-3K), and that administration of fluorescently labeled Aβ-42 to 5×FAD mice, leads to robust uptake into vesicular structures within spheroids (FIGS. 3L-3N). PLD3 overexpression in wildtype mice also led to occasional small axonal swellings with enlarged LAMP1-positive vesicles (FIG. 10I), suggesting that excessive PLD3 by itself can have detrimental effects independently of amyloidosis. Altogether, these data indicate that the accumulation of PLD3, observed in both mice and humans, is mechanistically linked with MVB abnormalities and the subsequent enlargement of axonal spheroids, which is compounded by the concurrent effect of PLD3 and Aβ accumulation within the same subcellular compartments.

### Example 5: PLD3 Deletion Reverses Axon Conduction Defects

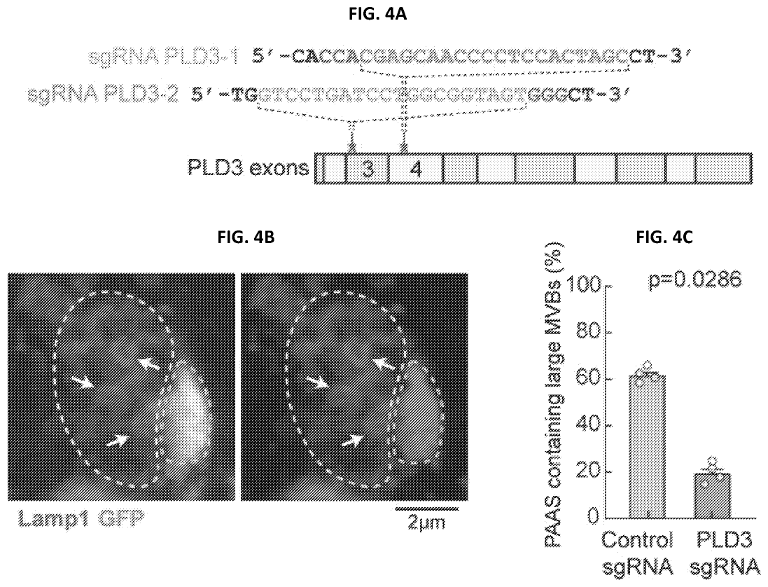

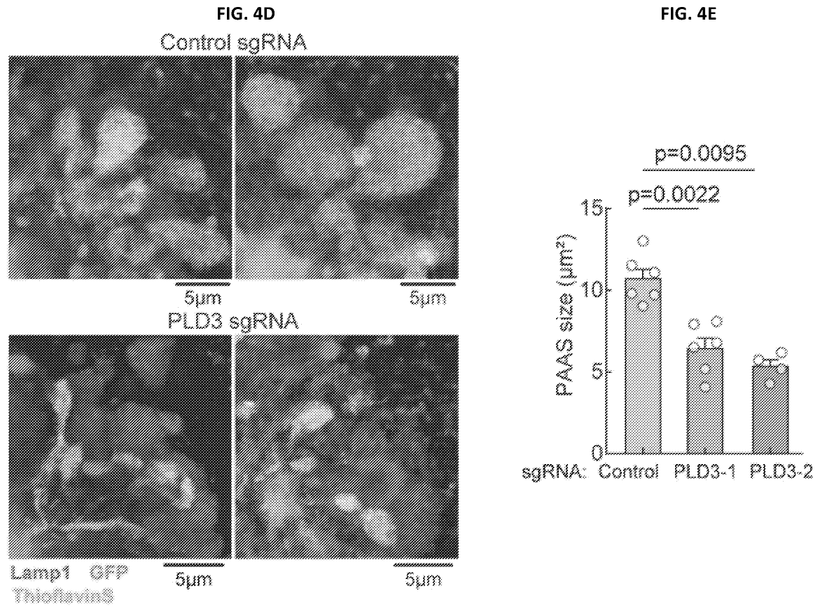

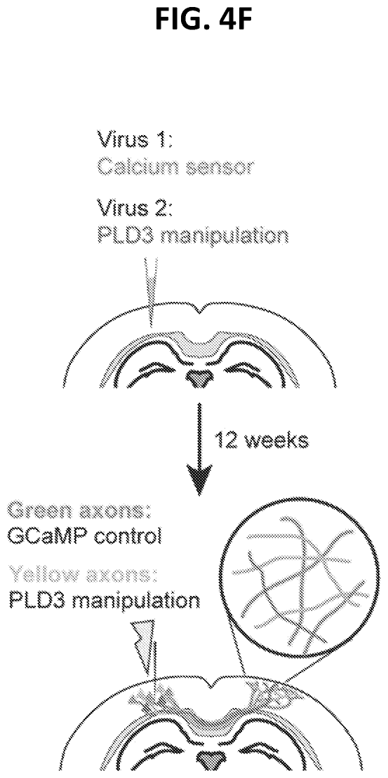

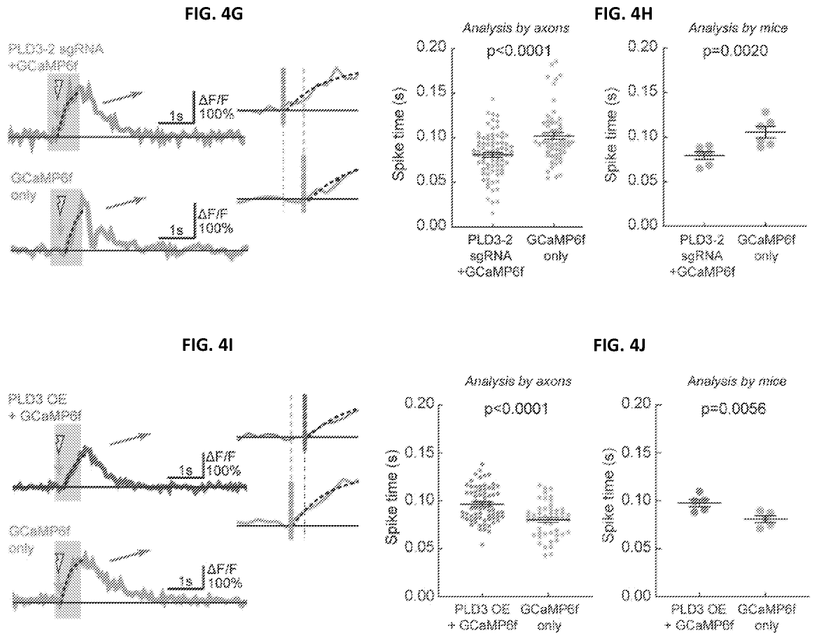

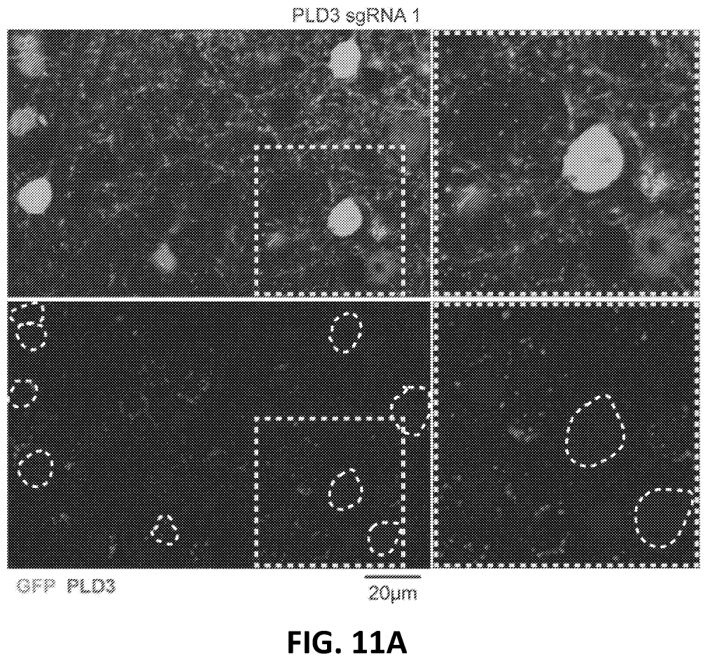

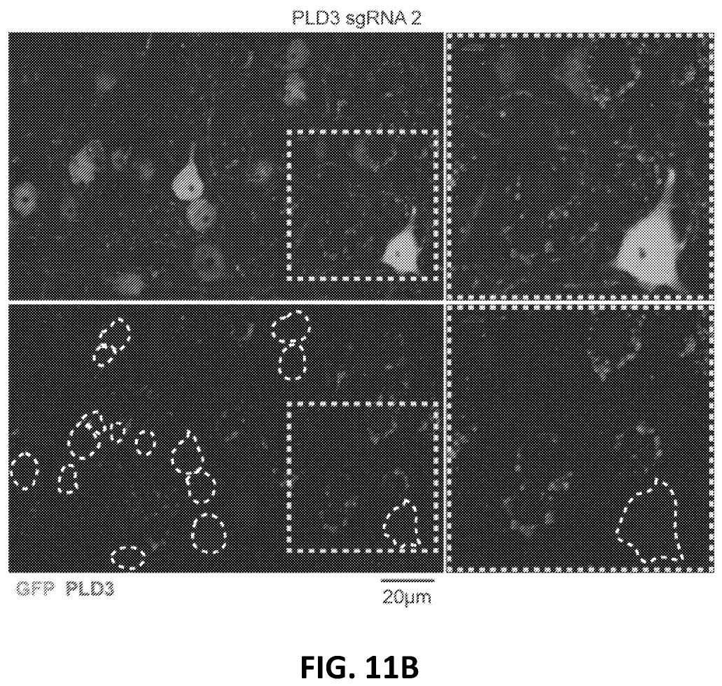

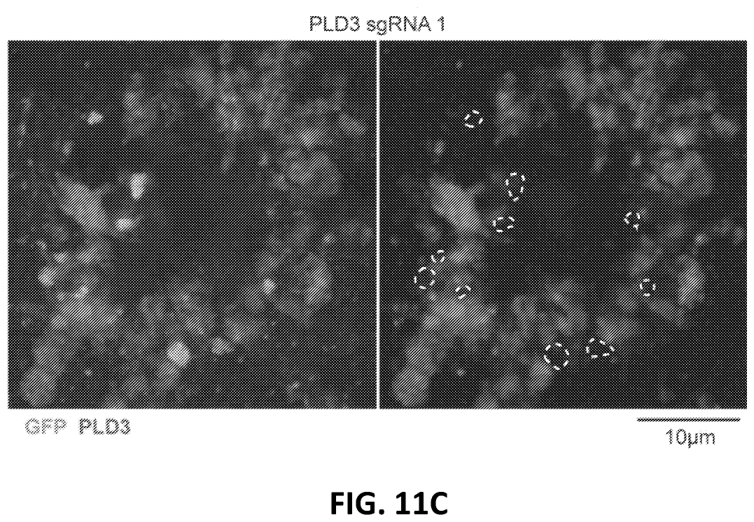

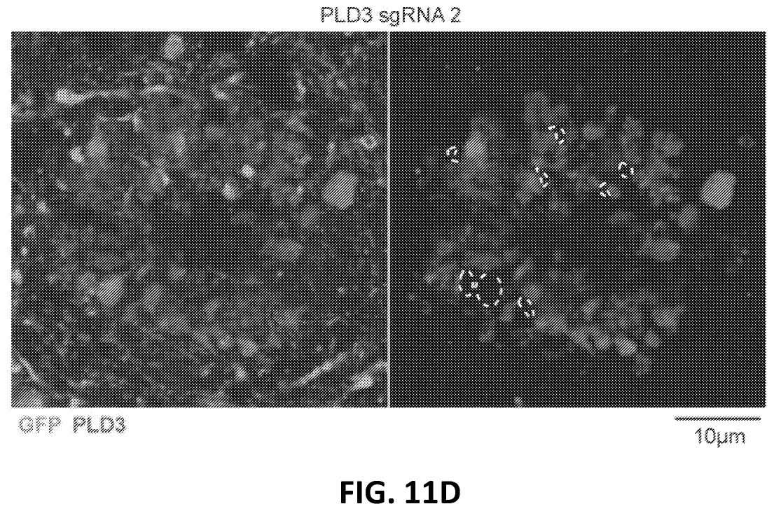

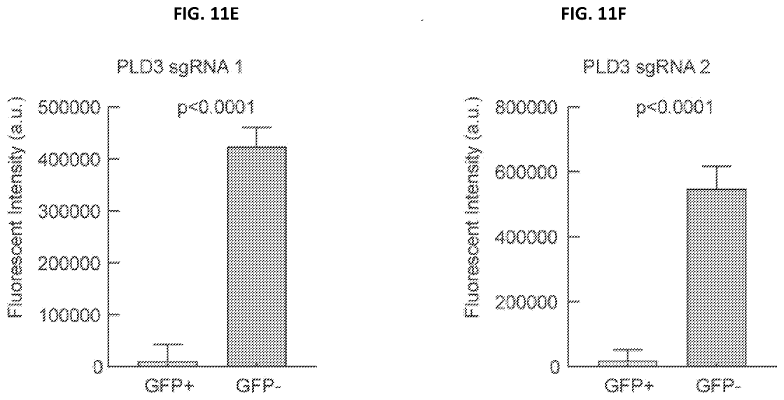

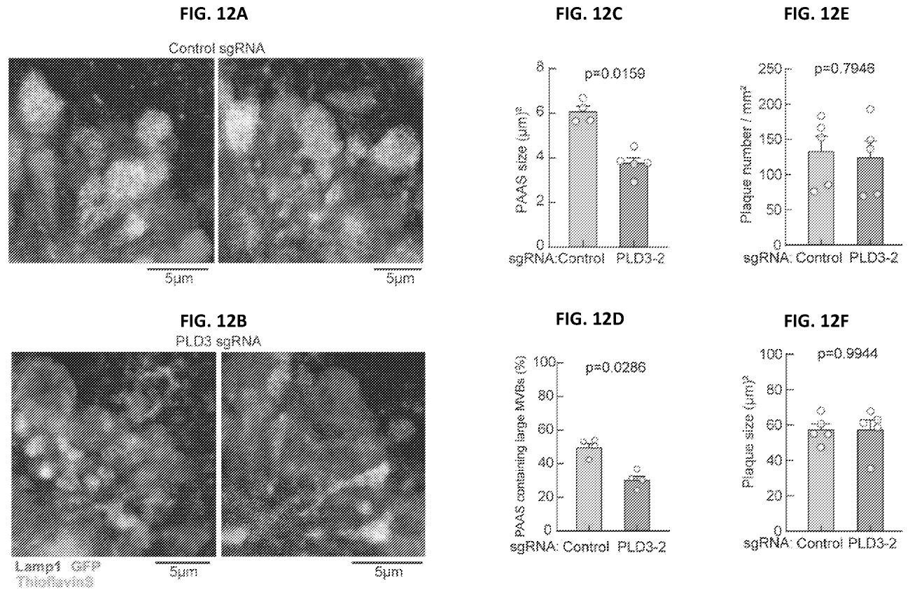

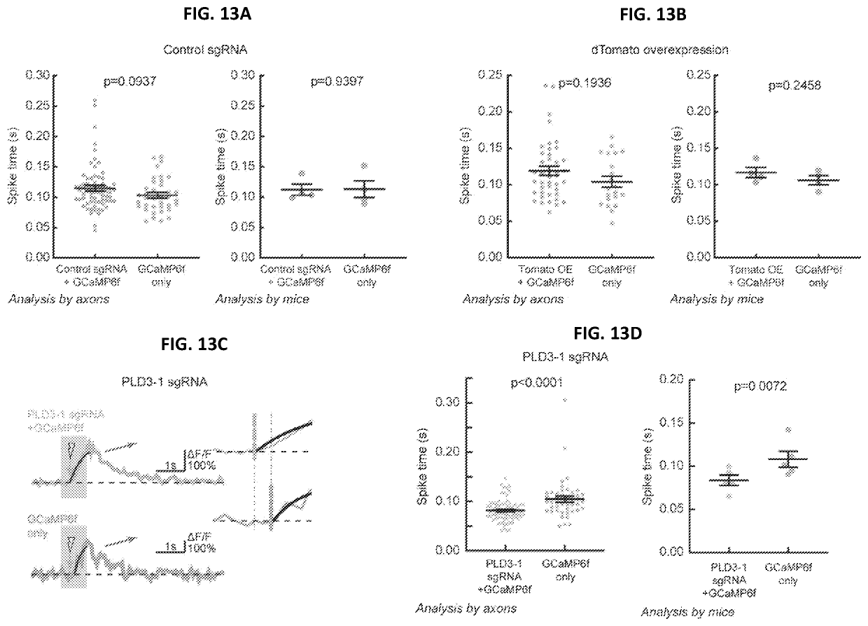

To test whether reducing PLD3 levels would ameliorate axonal spheroid pathology, PLD3 was deleted in neurons by AAV2-mediated CRISPR/Cas9 knockout in 5×FAD mice, using either of two single guide RNAs (sgRNAs) targeting different PLD3 exons (FIGS. 4A and 11A-11F). Treatment with both sgRNAs was found to lead to a marked decrease in the abundance of large MVBs (FIGS. 4B-4C and 12D) that was associated with an overall reduction in PAAS size, regardless of whether the treatment was initiated at 3 or 7 months of age in 5×FAD mice (FIGS. 4D-4E and 12A-12C). This data demonstrates that PLD3 deletion at early or later stages of amyloid deposition can decrease MVB accumulation in axons, leading to a marked attenuation in spheroid enlargement, without any changes in amyloid plaque number or size (FIGS. 12E-F).

To test whether deletion of PLD3 in neurons and the consequent reduction in spheroids size has a beneficial effect on axonal conduction, neurons were co-infected with AAV2-U6-sgRNA (PLD3)-CAG-Tomato-P2A-Cre to delete PLD3 and AAV9-Syn-GCamP6f to implement the Ca²⁺ imaging approach for measuring interhemispheric axonal conduction (FIG. 4F). This co-infection strategy allowed to image adjacent axons with or without PLD3 deletion within the same mouse and compare the Ca²⁺ rise times in contralateral cortex, following electrical stimulation of the ipsilateral hemisphere (FIG. 4F). Using the two sgRNAs, it was found that axons with PLD3 deletion had a marked improvement in the propagation of APs (FIGS. 4G-4H), which approached what is seen in control non-AD mice (FIG. 1K). In contrast, PLD3 overexpression had the opposite effect, with a slowing of conduction velocity observed in axons with increased PLD3, compared to adjacent controls (FIGS. 4I-4J). Together these data demonstrates that PLD3 reduction can reverse MVB abnormalities in axons near plaques, leading to reduced spheroid size and restored axonal conduction properties.

### Example 6: Reduction of Axonal Spheroids Improves Neural Circuit Function

To examine the impact of restoring conduction defects associated with axonal spheroids on neural circuit function, the present study focused on the basal forebrain (BF) nucleus of Meynert, which is a major source of cholinergic brain neurotransmission with extensive projections to the cortex and has been shown to degenerate in early stages of AD. Cortical projections from this nucleus are known to release acetylcholine to primarily activate interneurons and exert an overall inhibitory effect. Given the critical importance of this cholinergic circuit in cognitive function, the present study investigated the potential network effects of conduction deficits associated with PAAS and their potential reversibility by PLD3 modulation.

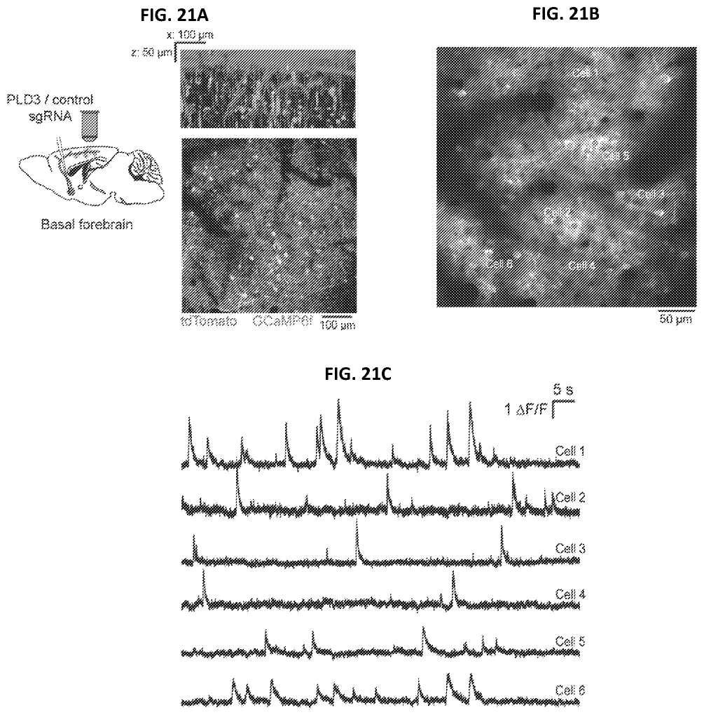

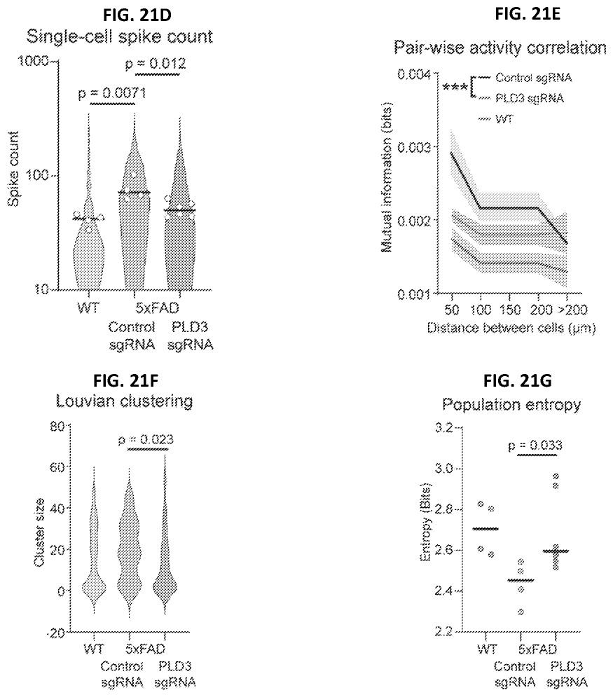

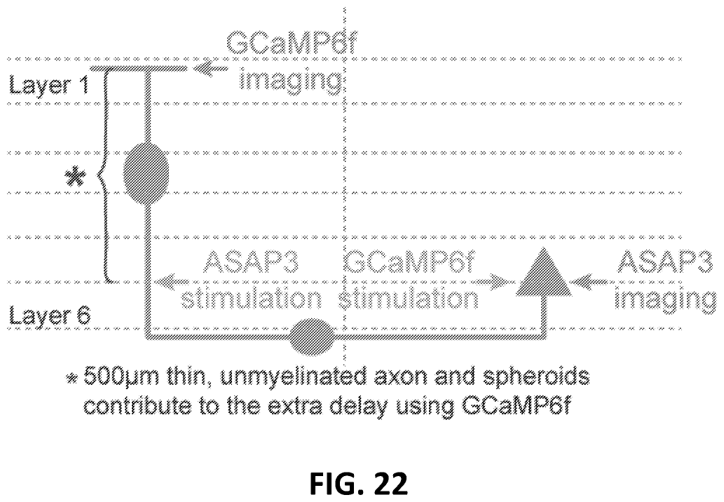

To achieve this, AAV2-U6-sgRNA (PLD3)-CAG-Tomato-P2A-Cre was injected to delete PLD3 in neurons of the basal forebrain in 7-month-old 5×FAD mice. To examine the effect of improved basal forebrain neurotransmission, the present study imaged spontaneous Ca²⁺ transients during awake resting sessions in neurons of layer ⅔ of the somatosensory cortex, previously infected with AAV9-Syn-GCamP6f (FIGS. 21A-21C). These neurons were within the immediate vicinity of projecting axons from basal forebrain (FIG. 21A, right panel). A higher proportion of hyperactive neurons in 5×FAD mice were observed (FIG. 21D). In addition, it was also found that 5×FAD mice showed increased correlated activity in neurons that were in close vicinity to each other (FIG. 21E), and displayed activity patterns with higher spatial-temporal similarities (FIGS. 21F-21G). These aberrant patterns of activity are predicted to markedly disrupt efficient information encoding. Importantly, PLD3 deletion in basal forebrain neurons led to a reduction in these aberrant activity patterns in cortical neurons to levels that were similar to WT controls (FIG. 21D-21G). Altogether, these data demonstrate that reducing action potential blockades in basal forebrain projection axons, which potentially improves neuromodulatory neurotransmission, can lead to a marked improvement on abnormalities in the patterns of activity of downstream cortical neurons.

### Example 7

The present study shows that hundreds of axons around each amyloid plaque develop spheroids and rather than being retraction bulbs from degenerating axons, these structures are stable for extended periods of time and therefore could significantly disrupt neural circuits. In vivo calcium imaging showed that when neurons are stimulated with a train of electrical pulses, spheroids cause axonal conduction blocks, which allow only a fraction of AP spikes to propagate, giving the appearance of conduction delays ranging from hundreds of milliseconds to seconds. Importantly, larger PAAS cause more severe conduction blocks, consistent with computational modeling showing that PAAS function as electrical capacitors that act as current sinks, and that PAAS size is a major determinant of the degree of conduction defects. These data suggests that the large number of plaques present in the AD brain have the potential to significantly affect neural networks by widespread disruption of axonal connectivity.

Consistent with this view, the quantitative histopathology analysis of human postmortem brain from AD or MCI patients showed that PAAS size and number correlates well with the degree of premortem cognitive decline. In AD, strategically located amyloid plaques could have deleterious effects in regions of the brain like the hippocampus where parallel compact axonal bundles follow a stereotyped projection path along a tri-synaptic loop 34. Memory formation may be particularly vulnerable to axonal conduction delays and blocks, given that hippocampal replay during memory consolidation is dependent on faithful spike reproducibility (also discussed herein in Example 12). Furthermore, axonal conduction defects could disrupt long-range connectivity between brain networks, as observed using resting state functional magnetic resonance imaging, and may also contribute to abnormal reaction time performance, a prominent feature of AD.

Mechanistically, the present study found that abnormally enlarged multivesicular bodies (MVBs) accumulate in axonal spheroids and their presence correlates with spheroid size. In addition, the present study found an increased presence of large MVBs within spheroids in older 5×FAD mice and in more severely impaired human AD patients, indicating that MVB accumulation may be a key feature of disease progression. Since spheroids in AD develop only in the vicinity of amyloid plaques, the MVB abnormalities found are likely to be amyloid dependent. Interestingly, robust endocytic activity was observed at axonal spheroids, which was associated with uptake of Aβ-42 oligomers into endosomal compartments within PAAS. Thus, internalization of Aβ from extracellular deposits may trigger endo-lysosomal abnormalities in axons. Indeed, Aβ-42 was present within MVBs at axonal spheroids, consistent with previous immunogold electron microscopy showing that in AD patients, the most prominent subcellular localization of Aβ-42 is within MVBs. These data suggest a mechanistic link between the accumulation of MVBs, due to their impeded maturation into lysosomes around plaques, and the subsequent enlargement of PAAS, which disrupt normal axonal conduction.

The endosomal sorting complex required for transport (ESCRT) machinery plays a major role in MVB biogenesis by regulating the formation of intraluminal vesicles (ILVs) within MVBs and the sorting of proteins into ILVs destined for degradation. In contrast to lysosomal resident proteins sorted to the limiting membrane of MVBs, PLD3, a potential risk factor for AD 19-21, is the only resident protein in mammals known to be sorted into IL Vs through the ESCRT pathway. Indeed, PLD3 was found to be present within IL Vs of large MVBs and highly enriched in axonal spheroids. This suggests that PLD3 may play a role in neuronal MVB maturation. Consistent with this, overexpression of PLD3 in neurons led to a marked enlargement and accumulation of MVBs and resulted in an overall increase in PAAS size. These data established a potential causal link between PLD3 accumulation in spheroids, MVB abnormalities and subsequent PAAS enlargement.

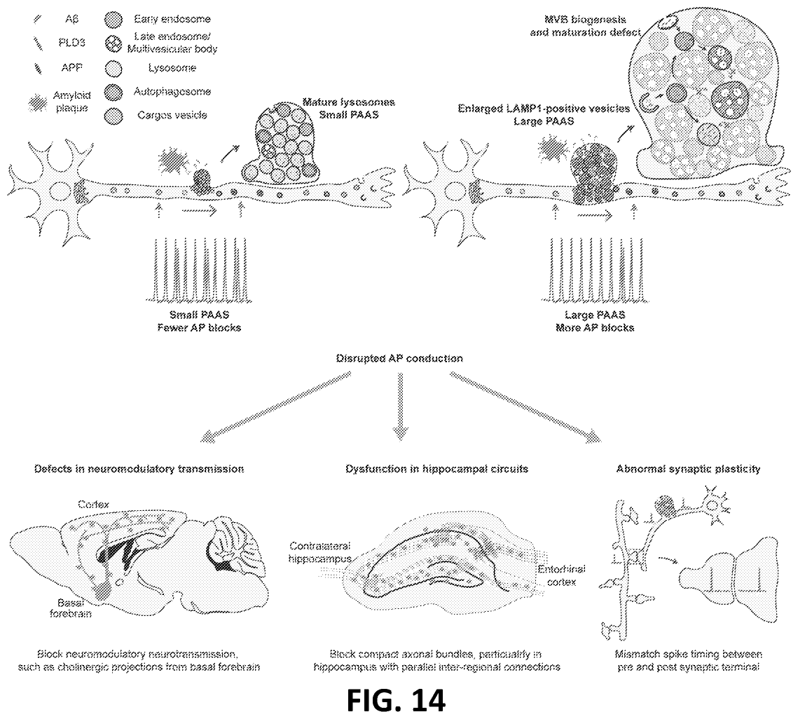

Deletion of ESCRT components can lead to enlarged endosomal compartments. Given the accumulation of PLD3 in axonal spheroids, it is possible that PLD3 leads to MVB enlargement by interfering with ESCRT machinery. In line with this, in wildtype mice, PLD3 overexpression led to enlargement of LAMP1-positive vesicles and formation of small axonal swellings. However, the dramatic enlargement of MVBs following PLD3 overexpression, predominantly occurs in PAAS which are in the vicinity of amyloid plaques. This suggests that extracellular Aβ is also critical for PLD3-induced MVB enlargement. Consistent with this, administration of Aβ-42 to cultured neurons has been shown to result in MVB enlargement. Together with the observation that Aβ-42 is actively endocytosed into PAAS and present within MVBs, PLD3 could work synergistically with Aβ-42 in the same subcellular compartment, leading to greater MVB abnormalities. On the other hand, given that APP and β-site amyloid precursor protein cleaving enzyme (BACE1) accumulate within PAAS, without wishing to be limited by any theory, intracellularly produced Aβ can also contribute to abnormalities in MVB biogenesis. Interestingly, APP is also sorted into IL Vs of MVBs through the ESCRT machinery, and deletion of ESCRT components promotes APP processing and increased intracellular Aβ 41. Thus, PLD3 and Aβ may constitute a vicious cycle in which axonal endocytosis and/or intracellularly produced Aβ, facilitates PLD3-induced MVB enlargement and accumulation (FIG. 14).

Given the finding that PLD3 overexpression increases PAAS size and worsens axonal conduction, reducing PLD3 can have a beneficial effect. Indeed, CRISPR/Cas9 neuronal knockout of PLD3 led to a reduction in the abundance of enlarged MVBs within spheroids and a decrease in PAAS size. Importantly, this led to a marked decrease in the frequency of axonal conduction blocks, thus improving interhemispheric connectivity. The effects of PLD3 deletion on PAAS size were seen in early as well as late stages of amyloidosis, indicating the potential for prevention as well as reversal of pre-existing spheroids.

While the focus of this investigation was on PLD3, in certain non-limiting embodiments, manipulation of other proteins in the endo-lysosomal pathway can lead to changes in PAAS size. However, given the negligible expression of PLD3 in non-neuronal cells (FIGS. 9A-9D) (despite mRNA presence in glial cells), this molecule could be a promising therapeutic target because global modulation of the endo-lysosomal pathway may negatively affect glial cells and their roles in controlling protein aggregation and amyloid brain accumulation. Furthermore, additional factors such as the glial microenvironment around plaques may also play a role in preventing PAAS formation. Thus, the interplay between intrinsic neuronal and extrinsic glial mechanisms may contribute to the formation and enlargement of PAAS and should be considered when designing therapies. Modulation of MVB biogenesis through PLD3 or other endo-lysosomal molecules could thus constitute a novel strategy for ameliorating PAAS pathology, independent of amyloid plaque removal.

The present findings reveal for the first time a cell-intrinsic neuronal mechanism that modulates the size of axonal spheroids and the consequent axonal conduction defects, with potentially important implications for AD-associated network dysfunction.

### Example 8: Methods

### Mice

5×FAD (34840-JAX, The Jackson Laboratory) mice were used in this study. Rosa26-LSL-Cas9 (026175, The Jackson Laboratory) mice were crossed with 5×FAD for CRISPR/Cas9-mediated gene deletion. The genotyping of 5×FAD mice was carried out following the instructions provided by The Jackson Laboratory. All animal procedures were approved by the Institutional Animal Care and Use Committee at Yale University.

### Antibodies and Reagents

The following primary antibodies were used in this study: anti-LAMP1 (DSHB, 1D4B), anti-GFP (Aves Labs. Inc. GFP-1020), anti-CathepsinD (Abcam, EPR3057Y, ab75852), anti-ATP6V0A1 (ThermoFisher Scientific, PA5-54570), anti-amyloid precursor protein (ThermoFisher Scientific, LN27, 13-0200), anti-PLD3 (Sigma-Aldrich, HPA012800), anti-beta amyloid 1-42 (Abcam, ab10148), anti-beta amyloid 1-42 (Abcam, mOC98, ab201061), anti-MAP2 (Abcam, ab5392), anti-Iba1 (Novus Biologicals, NB100-1028), anti-S100B (R&D Systems, AF1820), anti-Aldh111 (NeuroMab, P28037), FM1-43 (Life Technologies, F35355). All secondary antibodies used were conjugated with Alexa dyes from ThermoFisher Scientific. Thioflavin S (Sigma Aldrich, T1892) was used for staining amyloid plaques in fixed tissue. FSB (Santa Cruz, CAS 760988-03-2) was used for labeling plaques in live mice. To study spheroid endocytosis of extracellular Aβ, fluorescently labeled Aβ1-42 (AnaSpec) was used. Pitstop2 (Abcam, ab120687) was used to inhibit endocytosis.

### Adeno-Associated Virus (AAV) Production and Delivery

GCaMP6f and GCaMP6s viruses were purchased (UPenn Virus Core, AV-9-PV2822 and AV-9-PV2824; Addgene, #100837 and #100843). Customized AAV vectors for overexpression were constructed based on plasmid #28014 from Addgene, in which the GFP sequence was deleted and replaced by the customized sequences described below. In many cases where the virus transduced both a target protein and a fluorescent protein reporter, a GFP without the stop codon and P2A sequence was placed in front of the target protein sequence in the same open reading frame, as described previously (Yuan, P. et al., J Neurosci 36, 632-641 (2016)). The target proteins used in this study are:
tdTomato: sequence source http://www.tsienlab.ucsd.edu/Samples/PDF/tdTomato-map % 20&%20sequence.pdf, synthesized (Integrated DNA Technologies);
mCherry-SEpHluorin: sequence was cut from Addgene #32001;
LAMP1-GFP: LAMP-1 sequence was amplified from mouse brain mRNA, using the 5′ primer TGCGTCGCGCCATGGCGGCC (SEQ ID NO: 1) and 3′ primer GATGGTCTGATAGCCGGCGT (SEQ ID NO:2);
GFP-P2A-PLD3: PLD3 sequence was amplified from mouse PLD3 cDNA (GE open biosystem), using the 5′ primer ATGAAGCCCAAACTGATGTACCAGG (SEQ ID NO: 3) and 3′ primer TCAAAGCAGGCGGCAGGC (SEQ ID NO:4).

The sgRNA constructs for PLD3 deletion were cloned using plasmid #60229 from Addgene. The sequences of the sgRNAs are: PLD3 sgRNA 1: GTCCTGATCCTGGCGGTAGT (SEQ ID NO:5); PLD3 sgRNA 2: GCTAGTGGAGGGGTTGCTCG (SEQ ID NO:6); Control sgRNA: GGAAGAGCGAGCTCTTCT (SEQ ID NO: 7).

All the constructs were verified by DNA sequencing, and expression or deletion of the target proteins was tested with immunohistochemistry of the target protein.

AAV2 vectors were produced and purified following the procedures described previously (Grimm, D. et al., Mol Ther 7, 839-850 (2003)) using a two-plasmid helper free system (PlasmidFactory, Germany). Virus titer was determined by counting infection on HEK293 cells. AAV vectors were injected into the subarachnoid space in one hemisphere as previously described (Yuan, P. et al., J Neurosci 36, 632-641, (2016)). Total viral particles injected per mouse were approximately 107.

### Cranial Window Implant

8-month-old 5×FAD mice were anesthetized with ketamine/xylazine solution (100 mg/kg and 10 mg/kg, respectively) and hair was removed on the skull area. Buprenex (0.1 mg/kg), dexamethasone (2 mg/kg) and carprofen (5 mg/kg) was given subcutaneously at this point. The mouse was put on a heating pad during the surgery and anesthesia was checked periodically. Povidone-iodine solution was applied on the skin and cleaned with ethanol. And eye ointment was applied on the eyes. A small piece of skin was removed to expose skull, and the membrane tissue on the skull surface was removed by forceps. A 4 mm diameter circle was drilled on the contralateral hemisphere of virus infusion (rough location of the center is-2.5 mm from Bregma and 2.5 mm from midline). The skull was rinsed with sterile PBS periodically to avoid excessive heating. Skull was thinned in a circumferential area and then lifted with fine forceps without causing injury to the underlying pila surface. Gelfoam sponge (Pfizer Inc.) was used to absorb blood after lifting the skull. Using a pair of very fine forceps, the dura was removed within the circle area and a 4-mm cover glass was gently pressed on the brain surface and glued to the skull. A customized head-bar was glued (for acute imaging) or chronically implanted (with dental cement, for chronic imaging) on the skull. For chronic imaging, mice were put on a heating pad to recover after the surgery and given daily of buprenex (0.1 mg/kg) and carprofen (5 mg/kg) for 3 days. Imaging procedures started one month after the surgery.

### In Vivo Two Photon Imaging

Two-photon imaging was performed with a two-photon microscope equipped with a Ti-sapphire tunable laser (Spectra Physics), a Gallium arsenide phosphide (GaAsP) detector (Prairie technology) and a 20× water immersion objective (N.A. 1.0, Leica), or the Ultima Investigator multi-photon microscope (Bruker) with Insight X3 tunable ultrafast laser (Spectra Physics) and a 20× water immersion objective (N.A. 1.0, Olympus). GFP was excited at 920 nm; dTomato and tdTomato were excited at 920 nm/1045 nm; and FSB were excited at 850 nm. For chronic imaging, a location close to the center of the cranial window was selected as starting point and the blood vessel pattern was recorded. The coordinates of each region of interests were recorded as well. To relocate in the next imaging session, the starting point was relocated based on the recorded coordinates, and the field of view was adjusted to match the recorded blood vessel pattern.

### Abeta Preparation and Injection

Fluorescently tagged Abeta1-42 peptide (AnaSpec, 60480-1) was reconstituted in DMSO to a final concentration of 1 mg/mL. The solution was 1:10 (v/v) diluted in fresh artificial cerebrospinal fluid before being injected into the subacrachnoid space as described in Condell et al. (Nat Commun 6, 6176, doi: 10.1038/ncomms7176 (2015)). 10 μL of Abeta solution were injected for each mouse and the brain was harvested the next day. Brain tissue was prepared for immunohistochemistry.

### Calcium Imaging of Cortical Axons and Related Analysis

6-8 months 5×FAD mice were injected with GCaMP6 virus through the subarachnoid space on one hemisphere to label cortical neurons and measure local axonal conduction properties. For measurements of interhemispheric axonal conduction, GCaMP6f virus was injected stereotaxically with the following coordinates: Bregma (AP: −0.34, ML: 1.65, DV: 0.45, 2:0) 75 (Allen Mouse Brain Connectivity Atlas (2011)). After more than two weeks of the injection, an acute cranial imaging window was implanted on the contralateral hemisphere as described above. For stimulated calcium imaging, an additional opening on the skull was made on the ipsilateral side of the virus infusion. A glass electrode was inserted through this opening using a motorized micromanipulator and utilized for electrical stimulation.

The region of interest was identified under a two-photon microscope. GCaMP6-labele neurons were imaged through excitation at 920 nm wavelength. Limited field of view was used to improve sampling rate. GCaMP6s was imaged at 2 Hz and GCaMP6f was imaged at 10 to 20 Hz. Only axons that displayed spontaneous calcium transients at least once per minute were used for analysis. For stimulated calcium events, mice were anesthetized using 0.5% isoflurane. Stimulation trains of 2 ms pulses were delivered to the glass electrode at 50 Hz (18 ms interval) with 10 to 60 μA currents for 500 ms. The calcium responses within the imaging window were monitored upon stimulation. The stimulating electrode was adjusted to different depths within the cortex, to trigger responses in contralateral hemisphere axons. Three consecutive trials were acquired for each axon and the responses were averaged.

The raw GCaMP6 fluorescence intensity was normalized to ΔF/F for analysis. Images were then spatially smoothed with a 3×3 window. Several regions of interests (ROIs) were selected on each axon. The average ΔF before stimulations were used as baseline. For estimating the calcium rise time, the calcium trace from the event-specific peak to the first data point exceeding baseline was used as the rising phase of the event. This rising phase trace was fitted to an exponential equation: Y=1−exp (−k*(x−t)). The spike timing estimation (t0) was then calculated by extrapolating the x-intercept. For analysis of the spontaneous Ca²⁺ transients, the correlation coefficients between two ROIs chose on each axonal side of a particular spheroid were calculated, using the Pearson correlation coefficient.

### Calcium Imaging of Cortical Neuronal Networks and Related Analysis

AAV2 viruses encoding either PLD3 sgRNA or control sgRNA were injected stereotaxically into the basal forebrain of 6-8 months 5×FAD mice with the following coordinates: Bregma (AP: 0.62, 664 ML: 1.2, DV: 4.85, Z: 0) 75 (Allen Mouse Brain Connectivity Atlas (2011)). GCaMP6 virus was injected through the subarachnoid space on the ipsilateral hemisphere of basal forebrain injection to label cortical neurons.

The region of interest with intermingled projecting axons from basal forebrain and GCaMP6f-labeled cortical neurons was identified under a two-photon microscope. Calcium imaging was performed in cortical neurons of awake mice in the same region as those projecting forebrain axons. Spontaneous calcium activities of neurons were recorded at 25 Hz for 30 min.

For analyzing the cortical neuron activities, a rigid motion correction (Pnevmatikakis et al., J Neurosci Methods 291, 83-94, doi: 10.1016/j.jneumeth.2017.07.031 (2017)) was applied to the raw time lapse data and segmented the neuronal signal using an algorithm based on robust estimation (Inan et al. bioRxiv, doi: 10.1101/2021.03.24.436279 (2021)). Cell locations were defined as the centroid points from the spatial masks of each cell. The present study further estimated the neuronal spikes based on calcium traces as described in Deneux et al. (Nat Commun 7, 12190, doi: 10.1038/ncomms12190 (2016)). To calculate the pair-wise mutual information, the methodologies described in Timme et al. (eNeuro 5, doi: 10.1523/ENEURO.0052-18.2018 (2018)) were used. Mutual information from each cell pair in the same imaging session was grouped by the distances between neurons. To calculate neuron clusters with similar activity patterns, a graph theory-based community detection algorithms (Mucha et al., Science 328, 876-878, doi: 10.1126/science.1184819 (2010)) was used, with forced deterministic behavior for better reproducibility and determined the sizes of each cluster of cells identified in a single imaging session. To calculate population entropy, the present study followed the method described in Berry et al. (Journal of Statistical Mechanics: Theory and Experiment 2013, PO3015, doi: 10.1088/1742-5468/2013/03/p03015 (2013)). To account for different number of cells imaged, the present study drew a random subsample of 100 neurons from each mouse and calculated the population entropy. This process was repeated 100 times and the average results were recorded.

### Voltage Imaging and Analysis

AAV2-Syn-ASAP3 virus was injected stereotaxically into 6-8 months 5×FAD mice with the following coordinates: Bregma (AP: −0.34, ML: 1.65, DV: 0.45, ∠: 0) 75 (Allen Mouse Brain Connectivity Atlas (2011)). After more than two weeks of the injection, an acute cranial imaging window was implanted on the ipsilateral hemisphere as described above. An additional opening on the skull was made on the contralateral side of the virus infusion. A glass electrode was inserted through this opening using a motorized micromanipulator and utilized for electrical stimulation.

The region of interest was identified under a two-photon microscope. Line scan imaging of ASAP3-labeled neuronal soma were performed through excitation at 920 nm wavelength. The scanning speed was set to 1 kHz. Stimulation trains of 5 ms pulses were delivered at 10 Hz (95 ms intervals) with 10 to 100 uA currents for 1s.

Line scan images were used to extract the voltage response of ASAP3. On each line of the kymograph, the intensity of the pixels covering ASAP3-labeled neuronal soma was averaged to represent the membrane potential of the neuron at given time, which was used to generate the voltage response trace later. The response trace would show a big dip after each electrical stimulation pulse. The time of the dip indicated the antidromic AP time on the neuronal soma. To quantify the AP conduction failure, the present study performed fast Fourier transform (FFT) of the voltage response curve and took the 10 Hz (the same frequency as the electrical stimulation) component power as an indicator. The amplitude of the electrical current pulse was gradually increased; and FFT power-current amplitude could be plotted. The plot was fit with a logistic function: Y=a/(1+exp (−k*(x−b))). The current amplitude at the half-height of the logistic curve was defined as the threshold of the electrical stimulation.

### Spontaneous and Stimulated Calcium Imaging

6-8 months 5×FAD mice were injected with GCaMP6 virus through the subarachnoid space on one hemisphere to label cortical neurons and measure local axonal conduction properties. For measurements of interhemispheric axonal conduction, GCaMP6f virus were injected stereotaxically with the following coordinates: Bregma (AP: −0.34, ML: 1.65, DV: 0.45, ∠:0) 58 (Allen Mouse Brain Connectivity Atlas (2011)). After more than two weeks of the injection, an acute cranial imaging window was implanted on the contralateral hemisphere as described above. For stimulated calcium imaging, an additional opening on the skull was made on the ipsilateral side of the virus infusion. A glass electrode was inserted through this opening using a motorized micromanipulator and utilized for electrical stimulation.

The region of interest was located under a two-photon microscope. GCaMP6 were excited at 920 nm wavelength. Limited field of view was used to improve sampling rate. GCaMP6s was imaged at 2 Hz and GCaMP6f was imaged at 10 or 20 Hz. For imaging spontaneous activity, mice were imaged about three hours after initial ketamine/xylazine administration, and each axon was imaged for 10 minutes. Only axons with calcium transient at least once per minute were used for analysis. For stimulated calcium events, mice were anesthetized using 0.5% isoflurane. Spike trains of 2 ms pulses were delivered to the glass electrode at 50 Hz (18 ms interval) with 10 μA to 60 μA currents for 500 ms. The calcium response within the imaging window was monitored upon stimulation. The electrode was adjusted to different depths within the cortex, and the location that generated triggered responses in the axons of interest were used for experiments. Three consecutive trials of 5s or 10s imaging were acquired for each axon.

### Calcium Trace Analysis

The raw GCaMP6 fluorescence intensity was normalized to ΔF/F before analysis. Images were then spatially smoothed with a 3×3 window. Several regions of interests (ROIs) were selected on each axon. The average ΔF before stimulations were used as base line. For estimating the calcium rise time, the calcium trace from the event-specific peak to the first data point exceeding baseline was used as the rising phase of the event. This rising phase trace was fitted to an exponential equation: Y=1−exp (−k*(x−t)). The spike timing estimation (t0) was then calculated by extrapolating the x-intercept. For analysis of the spontaneous Ca²⁺ transients, the correlation coefficients between two ROIs chosen on each axonal side of the chosen spheroid was calculated using the Pearson correlation coefficient.

### Human Postmortem Brain Tissues

Formalin-fixed human postmortem brain tissue blocks were acquired from brain banks. Middle frontal gyrus, a cortical region affected in early stages of the disease59, was used for this study. Detailed information can be found in FIGS. 8A-8C, including twelve AD cases and six mild cognitive impairment cases. Cases were matched for age, gender, and ApoE genotype. For immunohistochemistry of human tissue, 30 μm-thick slices were prepared and treated with sodium citrate solution at 95 degrees for 45 minutes, before staining with primary antibodies for 3 days.

### Acute Organotypic Brain Slice Culture

Brain slice cultures were prepared from 8-month-old 5×FAD mice, following the protocol described in Hill et al. (Science 347, 543-548, doi: 10.1126/science. 1260088 (2015)). Briefly, the hippocampal region was dissected out in a sterile hood from anesthetized mice. Coronal sections around 300 microns thick were then manually cut and the slices were transferred onto a Millicell culture membrane (Fisher Scientific, PICM03050). The culture membrane was put in a six-well plate filled with 1 mL of the culture medium, and was placed in a 37-degree 5% CO₂ incubator. The present study examined the slice condition after 7 days with a light microscope. Healthy slices were then used for experiments. For measuring endocytosis, FM1-43 dye (ThermoFisher, T35356) or Dynasore (Tocris Bioscience) was added to the culture medium to the final concentration of 1 μM. Slices were incubated with the dye for 1.5 hours and then washed with fresh medium 2 times before fixing in 4% paraformaldehyde. For experiments blocking endocytosis, PitStop2 (Abcam, ab120687) was dissolved in dimethyl sulphoxide (DMSO) and added to the culture medium with different final concentrations of FM1-43 dye. Control groups used DMSO with no drug following the same dilutions.

### Plaque-Associated Axonal Spheroids Imaging and Quantification

Fixed tissue imaging was performed with a confocal microscope (Leica SP5 or Lecia SP8), and the images were taken with a 63× oil objective (N.A. 1.4, Leica). Individual PAAS size were measured by manually selecting outlines of individual bulbs based on LAMP1, APP or V0A1 immunohistochemistry. Multivesicular body (MVB)+ PAAS were defined as PAAS containing at least one LAMP1-positive ring structure. For classifying PAAS into neutral/acidic (using the genetically encoded pH sensor) and cathepsin D low/high groups, arbitrary thresholds of green/red fluorescence intensity of 0.75 and cathepsin D immunostaining fluorescence intensity of 100 were used using NIH imageJ/Fuji software.

### Expansion Microscopy

Expansion of brain sections were performed following conventional immunostaining. Brain sections were treated with Glutaraldehyde (GA; TCI Chemicals, G0068) and then subjected to gelation, digestion and expansion. Briefly, brain sections were first incubated with monomer solution (1×PBS, 2 M NaCl, 8.625% (w/w) sodium acrylate, 2.5% (w/w) acrylamide, 0.15% (w/w) N,N′-methylenebisacrylamide) at 4° C. for 45 min. Then transferred into a gel chamber and incubated in gelling solution (concentrated stocks (10% w/w) of ammonium persulfate (APS) initiator and tetramethyl-ethylenediamine (TEMED) accelerator added to the monomer solution for up to 0.2% (w/w) each and the inhibitor 4-hydroxy-2,2,6,6-tetramethylpiperidin-1-oxyl (4-hydroxy-TEMPO) added up to 0.01% (w/w) from a 0.5% (w/w) stock) at 37° C. for 1.5-2 hours for gelation. The gels were then fully immersed in proteinase solution (Proteinase K (New England Biolabs, P8107S) diluted 1:100 to 8 units/mL in the digestion buffer (50 mM Tris (pH 8), 1 mM EDTA, 0.5% Triton X-100, 1 M NaCl)) at 37° C. overnight. Digested gels were next placed in excess volumes of double deionized water (ddH₂O) for 25 min to expand. This step was repeated 3-5 times in ddH₂O, until the size of the expanding sample plateaued.

### Transmission Electron Microscopy

12-month-old 5×FAD mice were perfused with 4% PFA and the brain tissues were sectioned into 50 μm-thick slices with Vibratome (VT1000S, Leica). The slices were re-fixed in 2% glutaraldehyde in 0.1M cacodylate buffer (pH 7.4) for 1 hour, then post-fixed in 1% OsO₄ in the same buffer at room temperature for 1 hour. After en bloc staining with 2% aqueous uranyl acetate for 30 min, tissue was dehydrated in a graded series of ethanol to 100%, followed by propylene oxide and finally embedded in EMBed 812 resin. Tissue blocks were polymerized in 60° C. oven overnight. Thin sections (60 nm) were cut by a Leica ultramicrotome (UC7) and post-stained with 2% uranyl acetate and lead citrate. Sample grids were examined in a FEI Tecnai transmission electron microscope with accelerating voltage of 80 kV, digital electron micrographs were recorded with an Olympus Morada CCD camera and iTEM imaging software.

### AAV-Mediated Molecular Manipulations

AAV vectors were injected into ˜3-month-old and ˜7-month-old 5×FAD mice. AAVs were infused through subarachnoid space. Brain tissues were collected ˜1.5 months and ˜3 months after virus injection for treatments initiated at 3 and 7 months of age, respectively, and fixed with 4% paraformaldehyde. Brain slices of 50 μm thickness were prepared and stained with anti-LAMP1 antibody (DSHB, 1D4B) and Thioflavin S.

Imaging and quantification of PAAS were carried out according to Yuan, P. et al., Neuron 90, 724-739 (2016), and Yuan, P. et al., J Neurosci 36, 632-641 (2016). Briefly, tiled Z-stack images of the infected cortical regions were taken at zoom 1 at lum Z-steps. Individual plaques were segmented from the tiled images and blinded for the mouse and treatment information. For measurement of PAAS bulb size, the Z plane with the largest cross-section area of each individual spheroid was selected, and the cross-section area was measured using NIH Image J/Fiji software by manually selecting outlines of that cross-section based on virally expressed cytoplasmic GFP fluorescence.

### Computational Modeling

The modeling experiments of axon spheroids utilize methodologies according to Morse, T. et al., Front Neural Circuits 4, (2010). The chosen morphology parameters and ion channel distributions were held constant within each compartment for each simulation. The axon length was 566 microns (μm) and diameters were set from 0.1 to 0.9 μm. The spheroids were modeled as a “cylinder and stick”. The stick (5 μm length and diameter varying from 0.3 to 8.3 μm) was connected to the middle of the axon and on the other end to the cylinder (a bulb head) whose surface area varied from 0 to 7100 μm² (equivalent spherical diameter varied from 0 to 150 μm). In most simulations, the axon contained voltage gated sodium and potassium channels and a leak current. The densities of the voltage gated channels were varied from 0 to an amount 1.1 times as strong as needed to produce regenerative (sustained) action potentials in the axon. Channel conductances were set in the spheroids with the same (varying) strength as was present in the axon. One or more strong current injection(s) (0.2 ms 2 nA) was applied to one end of the axon to reliably evoke a single or multiple (input) AP(s) that subsequently propagated to the spheroids. Current injections just over threshold generated qualitatively similar results (larger diameter axons required more current to evoke an AP). The presence of (output) APs on the other side of the spheroids was checked by testing for the voltage exceeding 10 mV. One counted the number of output APs that were present in a 10 second simulation where either the single input AP occurred at, or the train of 20 Hz input APs began at 100 ms. The computer code according to NEURON simulation environment (Hines, M. L. et al., Neuroscientist 7, 123-135, (2001)) is made available at ModelDB: modeldb dot yale dot edu/187612.

### Statistics

For analyzing MVB presence, cathepsin D level and PH level in the axon spheroids, and spheroid bulb size after treatment, the specific numbers of plaques or spheroids measured from an individual human or mouse for each experiment can be found in each figure legend, and the average results were used as the representative outcome for that individual. The number of postmortem human tissues or mice was used as sample size in these cases. For analyzing difference in calcium rise time between region of interests (ROIs) along axon segments, the number of axons was used as sample size. This is because individual in vivo experiments had very few axons measured due to the necessary sparse labeling method used in these experiments. For analyzing inter-hemispheric calcium rise time delay, both the number of axons and mice were used as sample size. In the graphs, individual data points were shown. In the statistical comparisons, non-parametric tests were used unless otherwise justified. Specific tests used for each graph can be found in the corresponding figure legend. When more than two groups were considered and compared, corrections for multiple comparisons were performed as part of the post-hoc analysis. The statistics were calculated using GraphPad Prism software.

### Code Availability

All the custom codes for FIJI and MATLAB used in this study are deposited online: github dot com/PaulYJ/Axon-spheroid.

### Example 9: Estimating the Number of Axons Forming Spheroids Around Individual Amyloid Plaques

While it is likely that the number of affected axons around plaques varies significantly depending on age, brain region and other factors, it was attempted to obtain a ballpark estimation to understand the degree to which individual plaques can disrupt local neural circuits. The present study used the population distributions of their quantifications of the number of spheroids per axonal segment and the area of individual spheroids as seen in FIGS. 2A-2O, to estimate the total PAAS volume in single axon segments (step 1-4 below). The total volume of PAAS per plaque was quantified from about 1000 plaques in the cortex of 6-month-old 5×FAD mice based on LAMP-1 immunohistochemistry. The total PAAS volume per plaque divided by the total PAAS volume per axon segment indicates the number of axons affected per plaque.

### Step 1: Simulating the Number of PAAS in Individual Axon

As reported in FIGS. 6A-6F, the number of spheroids per axon follows a Gaussian distribution with a mean of 4.22 and SD of 1.95. To simulate the number of PAAS from a single axon, a random draw from the distribution was performed using the Norm.inv function in Excel:

=Norm.inv(Rand( ),4.22,1.95) (function 1)

Next, axons with at least one spheroid were kept for later calculation, using the following code:

=if(result of function 1>0.5,round(result of function 1),“ ”) (function 2)

10,000 resulting numbers were kept for later calculation.

### Step 2: Simulating the Volume of Individual Spheroids

Similar to step 1, a random draw from the Gaussian distribution of the log value of spheroid diameters was performed (FIGS. 6A-6F, mean of 0.48 and SD of 0.21). Again, the Norm.inv function in Excel was used:

=Norm.inv(Rand( ),0.48,0.21) (function 3)

And the resulting values were further transformed to volume:

$$= 4 / 3 * 3.14 * {( 0.5 * {10}^{∧} ( result of function 3 ) )}^{∧} 3 \qquad ( function 4 )$$

10,000 resulting numbers were kept for later calculation.

### Step 3: Simulating the Total Volume of all the Spheroids in Individual Axon Segments

To calculate the sum of volumes from multiple spheroids, a random number was first drawn from the step 1 results and that number was used to determine how many random numbers were drawn from the step 2 results. The total volume can then be calculated by summing these individual spheroids. This was achieved with Excel code:

=Sum(offset(first cell in results of step 2,Randbetween(1:9990),1,1,one result from result 1)) (function 5)

10,000 resulting numbers were kept for later calculation.

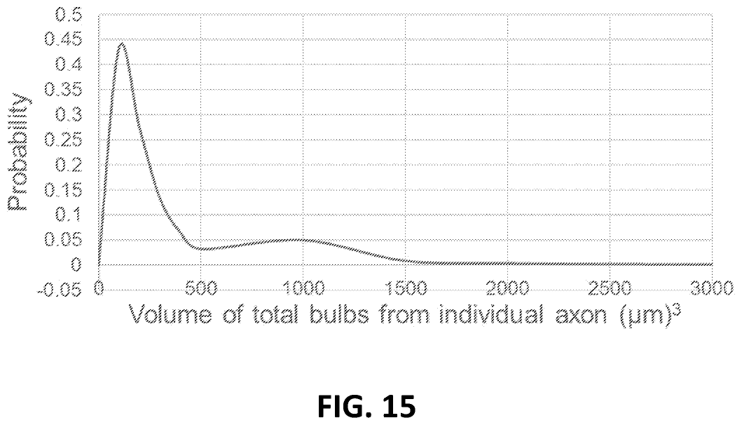

The distribution of these results (see FIG. 15) shows that the majority of individual PAAS volumes are below 500 μm³, and occasionally the volume can reach several thousands.

### Step 4: Simulation of the Sum of Several Axons

By taking a random sample from the results of Step 3 as an individual axon, several of these samples can then be summed to represent several axons.

Picking individual axons randomly from the distribution

=offset(first cell in results of step 3,Randbetween(1:9999),1,1,1) (function 6)

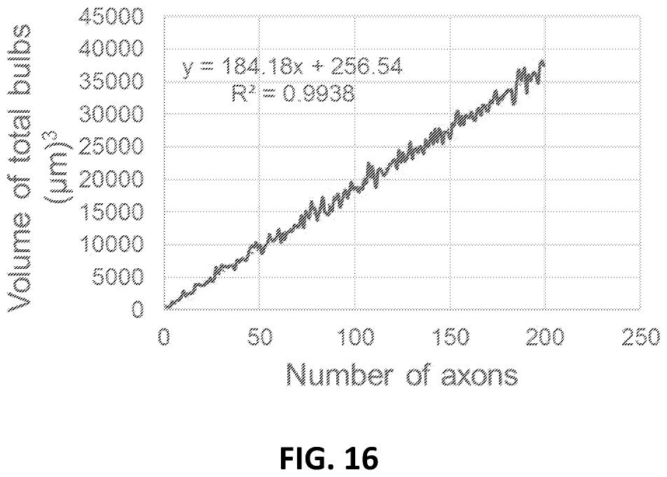

The summation from 2-200 axons was simulated from the results above. The results showed that the total volume of PAAS follows a positive linear correlation with the number of axons, with some noise. The noise almost disappeared when averaging the simulations 10 times (see FIG. 16).

Based on this result, each affected axon segment around plaques adds 184 μm³ PAAS volume on average to the total halo of PAAS around individual plaques.

### Step 5: Calculating the Number of Axons Affected Around Individual Amyloid Plaques

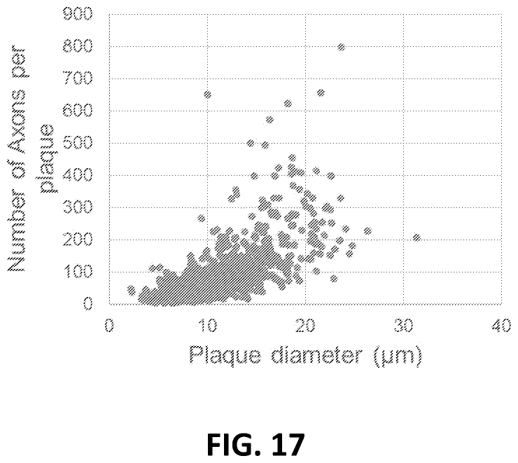

As described above, 1000 PAAS data from 6-month-old 5×FAD mice were used for this step. PAAS was quantified by measuring the LAMP-1 immunostained area at the center plane of individual amyloid plaques and extrapolated the volume assuming a spherical shape. And then, by dividing the calculated total PAAS halo volume around plaques by 184 (resulted from step 4), one can estimate the number of axons underlying this volume. The results of this plotting are shown in FIG. 17.

There is a non-linear, plaque size-dependent increase on the number of affected axons. The results were summarized below in Table 1, which shows the number of affected axons per plaque in relationship to plaque size.

TABLE 1

| | | Plaque | Plaque | Plaque |
|---|---|---|---|---|
| | Overall | Diameter < 10 | Diameter 10-20 | Diameter > 20 |
| Mean | 90.12 | 40.67 | 115.74 | 252.5 |
| Standard | 2.77 | 1.40 | 3.82 | 18.25 |
| Error | | | | |
| Median | 63 | 34 | 92 | 220.5 |
| Standard | 88.81 | 29.39 | 88.95 | 126.47 |
| Deviation | | | | |
| Minimum | 2 | 2 | 10 | 78 |
| Maximum | 797 | 265 | 649 | 797 |

### Example 10: Additional Analysis of Axonal Conduction Computational Modeling Results

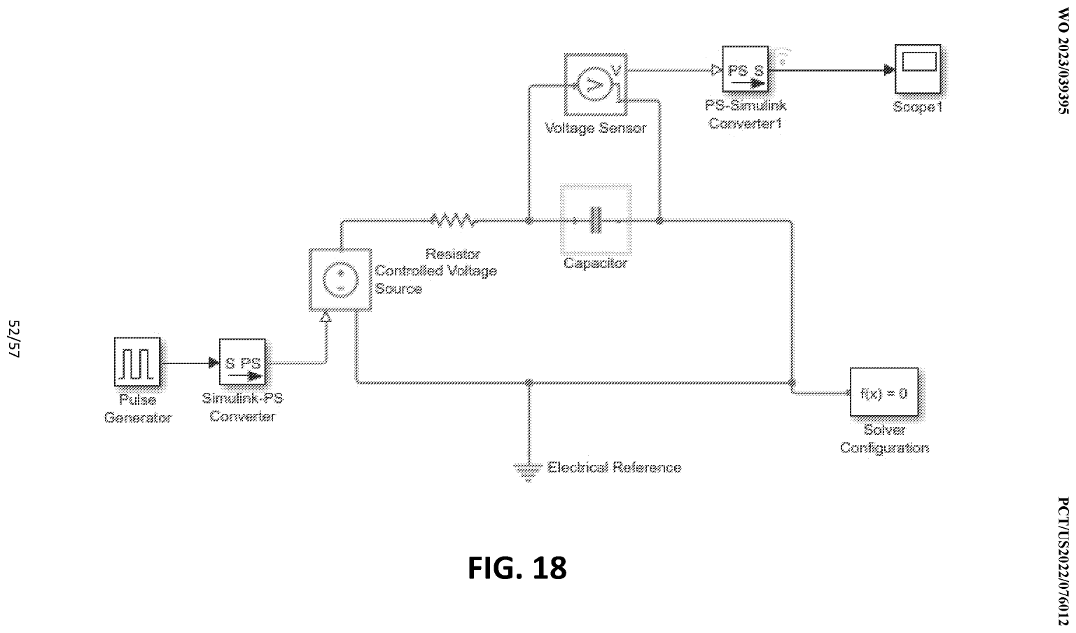

The computational modeling of action potential propagation along axonal segments near plaques showed that PAAS were electrically charged with every incoming action potential, and depending on whether the charging depolarizes the membrane potential and reaches the threshold for triggering an action potential, this charging will lead to a conduction delay or block. Such behavior can be fully recapitulated and perhaps more intuitively understood by considering the effect of a simple capacitor charging or discharging in an electric circuit (see FIG. 18).

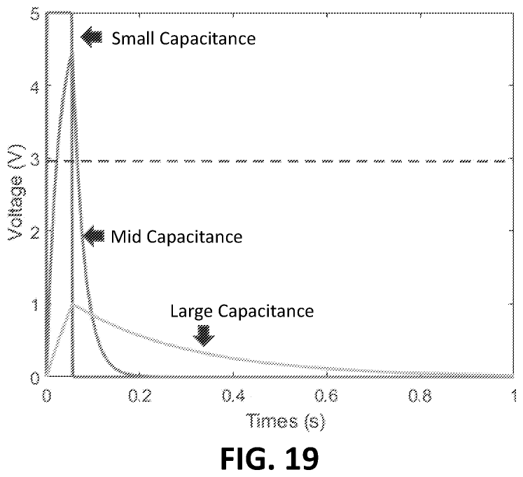

In this simple electric circuit simulated with MATLAB, a voltage source is controlled by a pulse generator to produce a voltage pattern of a simplified action potential. The circuit mimics the action potential propagating through the PAAS. Adjusting the size of the capacitor in this circuit, as shown below, recapitulates the basic findings in the experimental and mathematical modeling resulting from the PAAS effect on axonal conduction. If the pulse generator gives a single pulse, the voltage on the two ends of the capacitor changes over time in the manner as depicted in FIG. 19.

To symbolize the threshold for action potential, 3V was used as an arbitrary line for successful conduction (dash line). When the capacitance is small, it behaves as a closed wire, and the voltage follows the original pulse (“Small Capacitance”). In the case of middle capacitance (“Mid Capacitance”), the slow charging time leads to a delay for the signal to pass through the capacitor. When the capacitance is large, the charge will not reach the threshold (“Large Capacitance”). These are the categories of results the present study showed with biophysical modeling of action potential propagation through the axon segment.

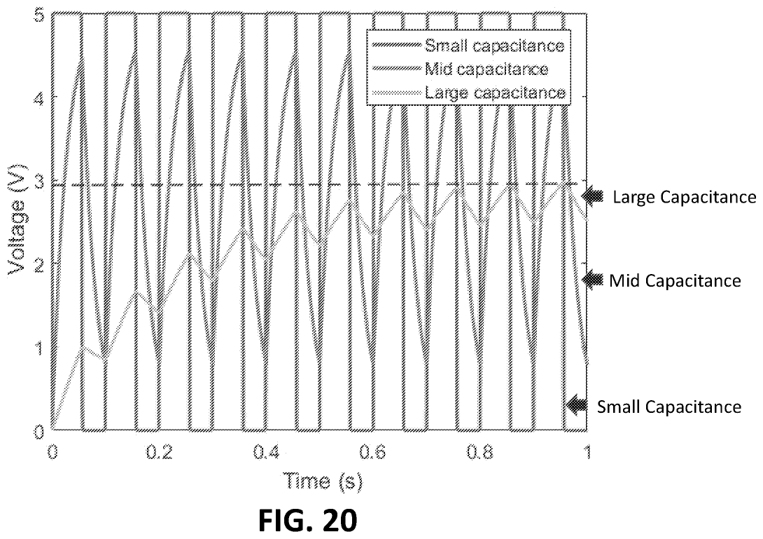

To simulate the situation with multiple pulses, the pulse generator was set to produce a 10 Hz signal. In the cases of small and middle capacitance, the capacitor can complete a charge-discharge cycle before the next pulse comes, resulting in 10 successful transmissions. The middle capacitance delays the transmission of each pulse. However, the capacitor produces an interesting behavior in the case of large capacitance. Each pulse charges the capacitor to a certain degree, but the time is too short for the capacitor to discharge completely before the next incoming pulse, leading to a small build-up of voltage with each pulse. In this particular case, it reaches the threshold after 10 pulses, resulting in a delay of action potential propagation of ˜1 second, and also reduces the output from 10 pulses to only 1 pulse (see FIG. 20). This is consistent with the modeling and experimental results (FIGS. 1A-1K and 6A-6F).

Therefore, it is useful to think of PAAS as an added capacitor in axons. One could use the known mathematical equations for capacitor charge-discharge process to think about the effect of PAAS on axon conduction.

$$Charge : Q = {CV}_{α} [ 1 - {e}^{- \frac{t}{R C}} ]$$

$$Discharge : Q = {CV}_{0} {e}^{- \frac{t}{RC}}$$

Q: total charge; C: capacitance; V: voltage; R: resistance; t: time.

As demonstrated above, these equations can describe the relationship between PAAS capacitance, firing rate and conduction abnormalities.

Importantly, the biophysical model indicates that the capacitance of PAAS is proportional to their size, and thus conduction delay or block should be more prevalent with larger PAAS surface areas.

### Example 11: Additional Discussion on the Impact of PAAS on Action Potential Conduction

To unambiguously demonstrate action potential delays or blockades by PAAS, the present study implemented ASAP3 voltage sensor to perform in vivo imaging. A single AP induces ˜20% change in GCaMP6 intensity, while ASAP3 only shows a ˜5% intensity decrease in live mouse brain. Because of its slow kinetics (lower decay time) and accumulation of intracellular Ca²⁺, GCaMP6 has a much larger ΔF/F during AP trains. Due to the negative directionality of change and small amplitude of the signal, ASAP3 is more prone to small vibration artifacts (triggered by heartbeat and breathing), therefore making single axon imaging very challenging. To overcome this issue, the present study took advantage of the back propagation of antidromic APs. Electrical stimulation was induced on the axon of transcallosal neurons, and two photon line scan imaging was performed on the soma of ASAP3 expressing neurons in the contralateral hemisphere layer V. This strategy allowed us to average multiple pixels on the scan line across the soma (FIG. 1M), which greatly improved signal-to-noise ratio and thus eliminated the limitations posed by movement.

One issue noticed when comparing interhemispheric conduction times using orthodromic Calcium imaging or antidromic voltage imaging was that the conduction times were not identical (FIGS. 1G and 1Q). However, this is unlikely to be related to the sensors themselves because when voltage imaging was compared with ASAP3 with Calcium imaging using GCaMP6 under the same antidromic stimulation conditions, it was found that the rise-time of single AP-induced calcium transients were similar to the spike of the ASAP3 transients (FIG. 1R), indicating that the Ca²⁺ rise time is a good surrogate of AP spike time.

Thus, the explanation for the difference in measurements in interhemispheric conduction with antidromic versus orthodromic stimulations relates the strategy for stimulation that is required in either condition. Specifically, during orthodromic GCaMP6 calcium imaging, the present study stimulate layer 5 cortical neuron cell bodies which include many transcallosal projecting cells. Calcium imaging was performed on the contralateral hemisphere superficial axons (˜100 μm below pial surface). In contrast, during antidromic stimulated voltage imaging with ASAP3, while the stimulation depth was very similar, the present study had to image the cell bodies of layer V neurons which are hundreds of microns deeper than the superficial axons imaged with GCaMP6. Therefore, the distance traveled by the action potential was much shorter for ASPA3 imaging, which likely explains the reduced conduction time measurements using this sensor, compared to GCaMP6.

### Example 12: Additional Discussion on the Impact of PAAS on Neural Networks Involved in Memory Formation

In certain embodiments, PAAS are particularly detrimental to neural processes that rely on temporally precise long-range coordination among brain regions, such as memory formation. Specifically, during system consolidation, hippocampus replays the representations of individual memorandums in temporally compressed neural spike sequences. And these sequential replays then guide distributed modification of synaptic connections, closely coupled with network oscillations such as sharp-wave ripples or pontogeniculooccipital waves. Two aspects of this process may be disrupted by PAAS: First, PAAS-mediated conduction delays or blockades could disrupt the faithful propagation of memory-encoding neural sequences in the brain. Similar to PAAS-mediated conduction disruption, experimentally disrupting the precise phase-lock synchronization of neural activities during system consolidation leads to failure of memory formation. In addition, PAAS could further interfere with the synaptic weight modification process, since precise timing of firing in axonal terminals and postsynaptic cells provides pivotal guidance of synaptic plasticity. Together, axonal conduction delay and block caused by PAAS may distort the neural processes underlying memory formation, potentially contributing to the anterograde amnesia in AD.

### ENUMERATED EMBODIMENTS

In some embodiments, the present invention is directed to the following non-limiting embodiments:

Embodiment 1: A method of reversing or preventing a formation and/or enlargement of an axonal spheroid, the method comprising: contacting a neuron affected by the formation or enlargement of the axonal spheroid with a compound that downregulates an expression level and/or an activity of PLD3.

Embodiment 2: The method according to Embodiment 1, wherein the axonal spheroid blocks or delays a propagation of an action potential (AP) along an axon of the neuron.

Embodiment 3: The method according to any one of Embodiments 1-2, wherein the formation and/or enlargement of axonal spheroids is associated with a neurodegenerative condition in a subject.

Embodiment 4: The method according to Embodiment 3, wherein the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia.

Embodiment 5: The method according to Embodiment 3, wherein the neurodegenerative condition is Alzheimer's disease.

Embodiment 6: The method according to Embodiment 5, wherein the axonal spheroid is associated with an amyloid plaque.

Embodiment 7: The method according to any one of Embodiments 1-6, wherein the compound that downregulates the expression level or the activity of PLD3 comprises at least one selected from the group consisting of: a small molecule inhibitor of PLD3, a protein inhibitor of PLD3, a nucleic acid that downregulates the expression level and/or activity of PLD3 by RNA interference, and/or an expression vector expressing the nucleic acid that downregulates the expression level and/or activity of PLD3 by RNA interference, a ribozyme that downregulates the expression level and/or activity of PLD3, and/or an expression vector expressing the ribozyme, an expression vector comprising an expression cassette, wherein the expression cassette expresses CRISPR components that downregulate the expression level and/or activity of PLD3 by CRISPR knockout or CRISPR knockdown, and a trans-dominant negative mutant protein of PLD3, and/or an expression vector that expresses the trans-dominant negative mutant protein of PLD3.

Embodiment 8: A method of treating, ameliorating, and/or preventing a neurodegenerative condition in a subject in need thereof, the method comprising: administering to the subject a compound that downregulates an expression level and/or activity of PLD3.

Embodiment 9: The method of Embodiment 8, wherein the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and/or Lewy Body dementia.

Embodiment 10: The method of any one of Embodiments 8-9, wherein the neurodegenerative condition is Alzheimer's disease.

Embodiment 11: The method of any one of Embodiments 8-10, wherein the subject is a human.

Embodiment 12: The method of any one of Embodiments 8-11, wherein the compound that downregulates the expression level and/or activity of PLD3 stops or reverses formation and/or enlargement of an axonal spheroid on an axon of a neuron cell.

Embodiment 13: The method of any one of Embodiments 8-12, wherein the compound comprises at least one selected from the group consisting of: a small molecule inhibitor of PLD3, a protein inhibitor of PLD3, a nucleic acid that downregulates the expression level and/or activity of PLD3 by RNA interference, and/or an expression vector expressing the nucleic acid that downregulates the expression level and/or activity of PLD3 by RNA interference, a ribozyme that downregulates the expression level and/or activity of PLD3, and/or an expression vector expressing the ribozyme, an expression vector comprising an expression cassette, wherein the expression cassette expresses CRISPR components that downregulate the expression level and/or activity of PLD3 by CRISPR knockout or CRISPR knockdown, and a trans-dominant negative mutant protein of PLD3, and/or an expression vector that expresses the trans-dominant negative mutant protein of PLD3.

Embodiment 14: The method of claim 13, wherein the compound comprises at least one selected from the group consisting of: the expression vector expressing the ribozyme, the expression vector comprising an expression cassette expressing the CRISPR components, and the expression vector that expresses the trans-dominant negative mutant protein, and wherein the expression vector comprises a viral vector.

Embodiment 15: The method of Embodiment 14, wherein the expression vector comprises an adeno-associated virus (AAV).

Embodiment 16: A pharmaceutical composition for treating a neurodegenerative condition in a subject, the pharmaceutical composition comprising: a compound that downregulates an expression level and/or activity of PLD3 in a neuron cell affected by the neurodegenerative condition; and a pharmaceutically acceptable carrier.

Embodiment 17: The pharmaceutical composition according to Embodiment 16, wherein the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia.

Embodiment 18: The pharmaceutical composition of Embodiment 17, wherein the neurodegenerative condition is Alzheimer's disease.

Embodiment 19: The pharmaceutical composition of Embodiment 17, wherein the compound comprises at least one selected from the group consisting of: a small molecule inhibitor of PLD3, a protein inhibitor of PLD3, a nucleic acid that down regulates the expression level and/or activity of PLD3 by RNA interference, and/or an expression vector expressing the nucleic acid that downregulates the expression level and/or activity of PLD3 by RNA interference, a ribozyme that downregulates the expression level and/or activity of PLD3, or an expression vector expressing the ribozyme, an expression vector comprising an expression cassette, wherein the expression cassette expresses CRISPR components that downregulate the expression level and/or activity of PLD3 by CRISPR knockout or CRISPR knockdown, and a trans-dominant negative mutant protein of PLD3, or an expression vector that expresses the trans-dominant negative mutant protein of PLD3.

Embodiment 20: The pharmaceutical composition of Embodiment 19, wherein the compound comprises at least one selected from the group consisting of: the expression vector expressing the ribozyme, the expression vector comprising an expression cassette expressing the CRISPR components, and the expression vector that expresses the trans-dominant negative mutant protein, and wherein the expression vector comprises a viral vector.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A method of reversing or preventing a formation and/or enlargement of an axonal spheroid, the method comprising:
contacting a neuron affected by the formation or enlargement of the axonal spheroid with a compound that downregulates an expression level and/or an activity of PLD3.

2. The method according to claim 1, wherein the axonal spheroid blocks or delays a propagation of an action potential (AP) along an axon of the neuron.

3. The method according to claim 1, wherein the formation or enlargement of axonal spheroids is associated with a neurodegenerative condition in a subject.

4. The method according to claim 3, wherein the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia.

5. The method according to claim 3, wherein the neurodegenerative condition is Alzheimer's disease.

6. The method according to claim 5, wherein the axonal spheroid is associated with an amyloid plaque.

7. The method according to claim 1, wherein the compound that downregulates the expression level or the activity of PLD3 comprises at least one selected from the group consisting of:
a small molecule inhibitor of PLD3,
a protein inhibitor of PLD3,
a nucleic acid that downregulates the expression level or activity of PLD3 by RNA interference, or an expression vector expressing the nucleic acid that downregulates the expression level or activity of PLD3 by RNA interference,
a ribozyme that downregulates the expression level or activity of PLD3, or an expression vector expressing the ribozyme,
an expression vector comprising an expression cassette, wherein the expression cassette expresses CRISPR components that downregulate the expression level or activity of PLD3 by CRISPR knockout or CRISPR knockdown, and
a trans-dominant negative mutant protein of PLD3, or an expression vector that expresses the trans-dominant negative mutant protein of PLD3.

8. A method of treating, ameliorating, or preventing a neurodegenerative condition in a subject in need thereof, the method comprising
administering to the subject a compound that downregulates an expression level or activity of PLD3.

9. The method of claim 8, wherein the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, or Lewy Body dementia.

10. The method of claim 8, wherein the neurodegenerative condition is Alzheimer's disease.

11. The method of claim 8, wherein the subject is a human.

12. The method of claim 8, wherein the compound that downregulates the expression level or activity of PLD3 stops or reverses formation or enlargement of an axonal spheroid on an axon of a neuron cell.

13. The method of claim 8, wherein the compound comprises at least one selected from the group consisting of:
a small molecule inhibitor of PLD3,
a protein inhibitor of PLD3,
a nucleic acid that downregulates the expression level or activity of PLD3 by RNA interference, or an expression vector expressing the nucleic acid that downregulates the expression level or activity of PLD3 by RNA interference,
a ribozyme that downregulates the expression level or activity of PLD3, or an expression vector expressing the ribozyme,
an expression vector comprising an expression cassette, wherein the expression cassette expresses CRISPR components that downregulate the expression level or activity of PLD3 by CRISPR knockout or CRISPR knockdown, and
a trans-dominant negative mutant protein of PLD3, or an expression vector that expresses the trans-dominant negative mutant protein of PLD3.

14. The method of claim 13,
wherein the compound comprises at least one selected from the group consisting of: the expression vector expressing the ribozyme, the expression vector comprising an expression cassette expressing the CRISPR components, and the expression vector that expresses the trans-dominant negative mutant protein, and
wherein the expression vector comprises a viral vector.

15. The method of claim 14, wherein the expression vector comprises an adeno-associated virus (AAV).

16. A pharmaceutical composition for treating a neurodegenerative condition in a subject, the pharmaceutical composition comprising:
a compound that downregulates an expression level or activity of PLD3 in a neuron cell affected by the neurodegenerative condition; and
a pharmaceutically acceptable carrier.

17. The pharmaceutical composition according to claim 16, wherein the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia.

18. The pharmaceutical composition of claim 17, wherein the neurodegenerative condition is Alzheimer's disease.

19. The pharmaceutical composition of claim 17, wherein the compound comprises at least one selected from the group consisting of:
a small molecule inhibitor of PLD3,
a protein inhibitor of PLD3,
a nucleic acid that down regulates the expression level or activity of PLD3 by RNA interference, or an expression vector expressing the nucleic acid that downregulates the expression level or activity of PLD3 by RNA interference,
a ribozyme that downregulates the expression level or activity of PLD3, or an expression vector expressing the ribozyme,
an expression vector comprising an expression cassette, wherein the expression cassette expresses CRISPR components that downregulate the expression level or activity of PLD3 by CRISPR knockout or CRISPR knockdown, and
a trans-dominant negative mutant protein of PLD3, or an expression vector that expresses the trans-dominant negative mutant protein of PLD3.

20. The pharmaceutical composition of claim 19,
wherein the compound comprises at least one selected from the group consisting of: the expression vector expressing the ribozyme, the expression vector comprising an expression cassette expressing the CRISPR components, and the expression vector that expresses the trans-dominant negative mutant protein, and
wherein the expression vector comprises a viral vector.
